SEQUENCE LISTING (7 sequences)

SEQ ID NO: 1 - DNA, 18 bp, Artificial Sequence
  Synthetic Polynucleotide
ttagggttagggttaggg

SEQ ID NO: 2 - DNA, 24 bp, Artificial Sequence
  Synthetic Polynucleotide
ttttttaatccgtcgagcagagtt

SEQ ID NO: 3 - DNA, 38 bp, Artificial Sequence
  Synthetic Polynucleotide
ttttttaatccgtcgagcagagttagggttagggttag

SEQ ID NO: 4 - DNA, 26 bp, Artificial Sequence
  Synthetic Polynucleotide
ttttttaatccgtcgagcagagttag

SEQ ID NO: 5 - DNA, 6 bp, Artificial Sequence
  Synthetic Polynucleotide
ttgggg

SEQ ID NO: 6 - DNA, 6 bp, Artificial Sequence
  Synthetic Polynucleotide
ttaggg

SEQ ID NO: 7 - RNA, 6 nt, Artificial Sequence
  Synthetic Polynucleotide
aauaaa

(19) United States
(12) Patent Application Publication
(10) Pub. No.: US 2009/0208932 A1
(43) Pub. Date: Aug. 20, 2009

(54) Methods Of Detecting An Analyte In A Sample

(76) Inventors: Edouard Collins Nice, Victoria (AU); Julie Anne Rothacker, Victoria (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/791,006

(22) PCT Filed: Nov. 16, 2005
(86) PCT No.: PCT/AU05/01742

§ 371 Date: Apr. 18, 2008

Related U.S. Application Data
(60) Provisional application No. 60/627,947, filed on Nov. 16, 2004.

(51) Int. Cl. C12Q 1/68; C07H 21/04

(52) U.S. Cl. 435/6; 536/23.1

(57) ABSTRACT

The invention relates to methods for detecting an analyte in a sample. The methods rely on the activity of polymerases upon polynucleotide substrates which are linked to a molecule, for example an antibody, which binds the analyte. Activity of the polymerases can be detected by the incorporation of suitably labelled nucleotides, and/or the incorporation of hapten conjugated nucleotides capable of binding a suitably labelled ligand of the hapten.

### FIELD OF THE INVENTION

The invention relates generally to methods for detecting an analyte in a sample. The methods rely on the activity of polymerases upon polynucleotide substrates which are linked to a molecule, for example an antibody, which binds the analyte. Activity of the polymerases can be detected by the incorporation of suitably labelled nucleotides, and/or the incorporation of hapten conjugated nucleotides capable of binding a suitably labelled ligand of the hapten.

### BACKGROUND OF THE INVENTION

Detecting, enumerating, and identifying low levels of a target analyte is a cornerstone of routine medical, industrial, and environmental diagnostics. For example, samples are analyzed to detect molecules from infectious agents, cancer cells, hormones, manufacturing contaminants, pollutants and agents used in bioterrorism.

Many different types of such detection methods are widely used in biomedical research and clinical laboratory medicine. Methods for detecting specific macromolecular species, such as proteins, have proven to be very valuable analytical techniques in biology and medicine, particularly for characterizing the molecular composition of normal and abnormal tissue samples. Examples of such detection methods include: immunoassays, immunochemical staining for microscopy, fluorescence-activated cell sorting (FACS) and the like.

Typically, a detection method employs at least one analytical reagent that binds to a specific target analyte and produces a detectable signal. These analytical reagents generally have two components: (1) a probe macromolecule, for example, an antibody, that can bind a target analyte with a high degree of specificity and affinity, and (2) a detectable label, such as a radioisotope or covalently-linked detectable molecule. In general, the binding properties of the probe macromolecule define the specificity of the detection method, and the detectability of the associated label determines the sensitivity of the detection method. The sensitivity of detection is in turn related to both the type of label employed and the quality and type of equipment available to detect the label.

The diagnosis of cancer in individuals has remained a difficult task to accomplish. Although some diagnostic markers are available that are assayable from blood or tissue samples, e.g. Carcinoembryonic Antigen (CEA), Alpha Fetoprotein (AFP) or Prostate Specific Antigen (PSA), the assays using these markers have not, to date, been markedly sensitive and/or specific of the presence of cancer in these individuals, as verified by other clinical diagnoses.

Immunological tests, or immunoassays, are ubiquitous in medical diagnostics. Based on the interaction of antibodies and the corresponding target analytes, immunoassays are used to detect a broad range of molecules ranging in size from small (e.g., a drug of abuse) to large (e.g., an HIV protein). Serological tests are immunological assays that, rather than testing directly for antigens, test for a host immunological response to previous exposure to the antigen—i.e., they test for the presence of host antibodies to the antigen. Numerous immunoassay systems are available ranging from large automated central laboratory systems to over-the-counter pregnancy tests. The tests cover a broad range of formats including agglutination assays, precipitin assays, enzyme-linked immunoassays, direct fluorescence assays, immuno-histological tests, complement-fixation assays, serological tests, immuno-electrophoretic assays, and rapid “strip” tests (e.g., lateral flow and flow through tests). One drawback of many immunological tests is that they are relatively insensitive. More specifically, numerous immunoassays, such as enzyme-linked immunoassays (ELISA), only result in the association of a single label moiety with the analyte. As a result, when levels of the analyte are low in the sample such methods do not produce a sufficiently strong signal to provide an accurate indication of the amount of analyte in the sample, and hence are prone to give false negative results.

At least one method that has been developed in an attempt to improve the sensitivity of immunoassays is “immuno-PCR” (Sano et al., 1992; Adler et al., 2003; Joerger et al., 1995; Sperl et al., 1995). This method relies on forming an analyte-antibody complex where the antibody is conjugated to a polynucleotide. The polynucleotide is then used as a template for the polymerase chain reaction (PCR), with the amplification products being used as an indicator for the level of the analyte in the sample tested. However, a major problem with this procedure is the well known deficiency of PCR exponentially amplifying a target polynucleotide and the resulting difficulties of obtaining an appropriate balance between sensitivity and accuracy to provide a true indication of the level of an analyte in a sample.

Another method that has been developed in an attempt to improve the sensitivity of immunoassays is that described by Zhang et al. (2001). Instead of relying upon PCR amplification of the polynucleotide conjugated to the antibody, Zhang et al. (2001) devised a method where the polynucleotide comprised a promoter for an RNA polymerase. Upon the RNA polymerase binding the promoter, an RNA which is complementary to the polynucleotide is transcribed. However, this procedure has the disadvantage that the product produced is RNA which is highly susceptible to degradation in many biological samples.

There is a need for further assay methods that can be used for the detection of an analyte in a sample.

### SUMMARY OF T INVENTION

The present inventors have devised assay procedures for detecting an analyte in a sample. The assay of the invention results in the formation of a detectable complex which comprises more than one label moiety associated with the analyte.

In a first aspect, the present invention provides a method of screening for the presence or absence of an analyte in a sample, the method comprising

i) exposing the sample to a first compound that binds the analyte to form an analyte-first compound complex,

ii) exposing the analyte-first compound complex to a second compound which binds the analyte-first compound complex to form an analyte-first compound-second compound complex, wherein the second compound comprises a polynucleotide,

iii) exposing the analyte-first compound-second compound complex to a polymerase under conditions which allow either a) the polymerase to extend the polynucleotide, or b) the polymerase to synthesize a complementary strand of the polynucleotide, and

iv) detecting the product of parts a) or b) of step iii), wherein part b) of step iii) does not comprise using the complementary strand as a template for further polynucleotide synthesis.

In a second aspect, the present invention provides a method of screening for the presence or absence of an analyte in a sample, the method comprising

i) exposing the sample to a second compound that binds the analyte-second compound complex to form an analyte-second compound complex, wherein the second compound comprises a polynucleotide,

ii) exposing the analyte-second compound complex to a first compound which binds the analyte form an analyte-first compound-second compound complex,

iii) exposing the analyte-first compound-second compound complex to a polymerase under conditions which allow either a) the polymerase to extend the polynucleotide, or b) the polymerase to synthesize a complementary strand of the polynucleotide, and

iv) detecting the product of parts a) or b) of step iii),

wherein part b) of step iii) does not comprise using the complementary strand as a template for further polynucleotide synthesis.

The detection of polymerase products produced in the assay indicates the presence of the analyte under assay. The products can also be quantitated, and the quantity of the products can be correlated with the quantity of the analyte assayed. Thus, the methods of invention can be used for detecting, or for detecting and quantitating, an analyte in a test sample.

In a particularly preferred embodiment, the polymerase extends a single stranded polynucleotide or a single stranded overhang of a partially double stranded polynucleotide.

Not only does the above embodiment have the advantage of not requiring the addition of a primer, there is the added advantage that the polynucleotide linked to an antibody (for example) can be relatively short. Thus, in a further preferred embodiment, the polynucleotide is less than about 100 nucleotides in length, more preferably less than about 75 nucleotides in length, more preferably less than about 50 nucleotides in length, more preferably less than about 40 nucleotides in length, more preferably less than about 30 nucleotides in length, more preferably less than about 20 nucleotides in length.

In another embodiment, the method does not comprise the use of a primer which hybridizes to the polynucleotide.

Examples of polymerases which extend a single stranded polynucleotide or a single stranded overhang of a partially double stranded polynucleotide include, but are not limited to, poly(A)polymerase, T4 RNA ligase, telomerase and terminal transferase. Preferably, the polymerase is a telomerase or terminal transferase.

The telomerase can be isolated from a natural source or produced recombinantly. Furthermore, the telomerase can be from an organism that produces such molecules, or a variant/derivative/mutant thereof which possesses telomerase activity.

The present inventors have found that cancer cells, particularly human cancer cells, provide a convenient source of telomerases for use in the methods of the invention. Thus, in a preferred embodiment, the telomerase is obtained by lysing cancer cells producing the telomerase. Notably, it has been determined that there is no need to purify the telomerase from other components of the cell lysate before being used in the methods of the invention. As a result, it is preferred that no procedures are performed to separate the telomerase from other components of the lysed cells.

As indicated above, it is preferred that the polymerase is capable of extending the polynucleotide, or synthesizing a complementary strand of the polynucleotide, in the absence of a suitable primer. However, in some embodiments which utilizes enzymes such as Taq polymerase it will be required to include a suitable primer which hybridizes the polynucleotide under the reaction conditions and acts to initiate synthesis of a complementary strand. Suitable primers can readily be designed based on the sequence of the polynucleotide. Such primers are typically small, being at least about 12 nucleotides in length, at least about 15 nucleotides in length, at least about 18 nucleotides in length, at least about 21 nucleotides in length, or at least about 24 nucleotides in length. In a particularly preferred embodiment, the primer is linear. In another particularly preferred embodiment, the primer is not circular.

In another embodiment the polymerase is a DNA polymerase. Suitable DNA polymerases include, but are not limited to, Taq polymerase, bacteriophage T4 polymerase, bacteriophage T7 polymerase, and E. coli DNA polymerase I Klenow fragment.

In a further embodiment, in the instance where the polymerase is an RNA polymerase the polynucleotide does not comprises a promoter region which the RNA polymerase uses to primer RNA transcription.

Preferably, the first compound and/or second compound is attached to a solid support. Any suitable solid support known to the skilled person can be used. Examples include, but are not limited to, magnetic beads, biosensor chips, wells of a microtiter plate, dipsticks, and microarray slides.

In another embodiment, step iii) is performed in the presence of at least one detectably labelled nucleotide. Any suitable labelled nucleotide known to the skilled addressee can be used. Examples include, but are not limited to, radioactive isotopes, fluorescent labels, chemiluminescent labels, bioluminescent labels and enzyme labels.

In a further embodiment, step iii) is performed in the presence of at least one hapten conjugated nucleotide, wherein the hapten is capable of binding a ligand. Examples of suitable haptens include, but are not limited to, cysteine, lysine, serine, biotin, avidin and streptavidin.

Preferably, the ligand is detectably labelled. Any suitable detectable label known to the skilled addressee can be used. Examples include, but are not limited to, radioactive isotopes, fluorescent labels, chemiluminescent labels, bioluminescent labels and enzyme labels.

Preferably, step iii) further comprises exposing the products of polymerase activity to the detectably labelled ligand.

Preferably, the detectable label is an enzyme. Any suitable labelled enzyme known to the skilled addressee can be used. Examples include, but are not limited to, β-galactosidase, luciferase, alkaline phosphatase, neuraminidase, and horse-radish peroxidase. Preferably, the products of polymerase activity of step iii) is a complex at least comprising polynucleotides with hapten conjugated nucleotides incorporated therein, wherein at least some of the haptens are bound to an enzyme labelled ligand, and wherein step iv) comprises exposing the products of step iii) to conditions which allow the enzyme to produce a detectable signal.

Preferably, the detectable signal is produced by the enzyme reacting with a substrate. Suitable substrates for use in the methods of the invention are known to those skilled in the art. Examples include, but are not limited to, luminol or acridan. Co-factors for enzyme activity may also be required, such as the presence of hydrogen peroxide in reactions comprising luminol as a substrate. Furthermore, a molecule which enhances the detectable signal can be provided. Such molecules are known in the art and include, but are not limited to, p-iodophenol or p-phenylphenol.

Preferably, the detectable signal is luminescence or fluorescence.

Each of the methods of invention will most likely include steps of removing unbound analytes, compounds, substrates, etc. Examples of such steps include, but are not limited to:
step i) of the first aspect further comprises washing the analyte-first compound complexes to remove unbound first compound,
step i) of the second aspect further comprises washing the analyte-second compound complexes to remove unbound second compound,
step ii) further comprises washing the analyte-first compound-second compound complexes to remove unbound first and/or second compound,
removing unincorporated detectably labelled nucleotides, and/or
removing unincorporated detectably labelled ligands.

The first compound can be any compound which specifically binds the analyte in the sample. In a preferred embodiment, the first compound is a protein. More preferably, the first compound is an antibody.

The second compound comprises a polynucleotide and specifically binds an analyte, and/or first compound-analyte complex. Preferably, the second compound is a protein-polynucleotide conjugate. More preferably, the second compound is an antibody-polynucleotide conjugate. Furthermore, it is preferred that the second compound binds the analyte.

In a preferred embodiment, the analyte is a marker of a disease state. More preferably, the disease state is selected from, but not limited to, cancer and an infection.

Suitable analytes which can be detected using the methods of the invention include organic and inorganic molecules, including biomolecules. In a preferred embodiment, the analyte is a protein, a peptide, or a small molecule such as small organic molecule.

In one particularly preferred embodiment, the method comprises

i) exposing the sample to a first compound that binds the analyte to form an analyte-first compound complex, wherein the first compound is attached to a solid support,

ii) exposing the analyte-first compound complex to a second compound which binds the analyte to form an analyte-first compound-second compound complex, wherein the second compound comprises a polynucleotide which can be extended by a telomerase,

iii) exposing the analyte-first compound-second compound complexes to a telomerase in the presence of at least one hapten conjugated nucleotide under conditions which allow the telomerase to extend the polynucleotide,

iv) exposing the products of telomerase activity to a enzyme labelled ligand under conditions which allow the hapten to bind to the ligand,

v) incubating the polynucleotide-hapten-ligand-enzyme complex in the presence of a substrate of the enzyme, and

vi) detecting a detectable signal produced by the activity of the enzyme on the substrate.

In another particularly preferred embodiment, the method comprises

i) exposing the sample to a first compound that binds the analyte to form an analyte-first compound complex, wherein the first compound is attached to a solid support,

ii) exposing the analyte-first compound complex to a second compound which binds the analyte to form an analyte-first compound-second compound complex,

wherein the second compound comprises a polynucleotide which can be extended by a terminal transferase,

iii) exposing the analyte-first compound-second compound complexes to a terminal transferase in the presence of at least one hapten conjugated nucleotide under conditions which allow the terminal transferase to extend the polynucleotide,

iv) exposing the products of terminal transferase activity to a enzyme labelled ligand under conditions which allow the hapten to bind to the ligand,

v) incubating the polynucleotide-hapten-ligand-enzyme complex in the presence of a substrate of the enzyme, and

vi) detecting a detectable signal produced by the activity of the enzyme on the substrate.

In a further aspect, the present invention provides a method of screening for the presence or absence of an analyte in a sample, the method comprising

i) exposing the sample to a first compound that binds the analyte to form an analyte-first compound complex,

ii) exposing the analyte-first compound complex to a second compound which binds the analyte-first compound complex to form an analyte-first compound-second compound complex,

iii) exposing the analyte-first compound-second compound complex to a third compound which binds the second compound, wherein the third compound comprises a polynucleotide,

iv) exposing the analyte-first compound-second compound-third compound complex to a polymerase under conditions which allow either a) the polymerase to extend the polynucleotide, or b) the polymerase to synthesize a complementary strand of the polynucleotide, and

v) detecting the product of parts a) or b) of step iv),

wherein part b) of step iii) does not comprise using the complementary strand as a template for further polynucleotide synthesis, and wherein the first and/or second compound is bound to a solid support.

In this aspect of the invention the “third compound” can be considered as a “universal” reagent which could be used in methods for the detection of any analyte. In this aspect, it is preferred that the second compound is an antibody and the third compound comprises an antibody, wherein the second compound (antibody) is from a first animal species and the antibody of the third compound binds the second compound (antibody) and is from a different animal species. Such antibodies, and methods for the production thereof, are well known in the art. For example, the second compound (antibody) can be derived from the immunization of mice with the analyte, and the third compound comprise an anti-mouse anti-IgG rabbit antibody. In another example, the second compound (antibody) can be derived from the immunization of rabbits with the analyte, and the third compound comprise an anti-rabbit anti-IgG goat antibody.

The advantage of having an assay that uses a “universal” reagent is that it not necessary to produce polynucleotide conjugates for the detection of different analytes. For instance, two different antibodies could be produced from mice which independently bind the known cancer markers CEA and PSA respectively, however, the same polynucleotide conjugated anti-mouse anti-IgG rabbit antibody could be used in separate methods for the detection of these analytes.

Following the utilization of the polymerase, the synthesized or extended polynucleotide strand may be dissociated from the complex, however, the detection of the synthesized or extended strand in any aspect of the invention can readily proceed without the need to perform a dissociation step.

One can control the sensitivity of the methods of the invention to some degree by varying the concentration of key factors, such as the concentration of compounds which bind the analyte, concentration of the nucleotide precursors, ratio of labelled to unlabelled nucleotide precursors, the amount of polymerase, and the detection method. A specific assay can be optimized by methods known to those of skill in the art.

Also provided is a protein-DNA conjugate, wherein the DNA comprises a sequence which can bind, and be extended by, a telomerase. Such a conjugate can be used in the methods of the invention. Preferably, the protein is an antibody.

In another aspect, the present invention provides a kit comprising the protein-DNA conjugate of the invention.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1—Schematic representation of an embodiment of the method first aspect of the invention. In this example, the first compound is an antibody bound to a magnetic bead. The magnetic beads allow the complexes formed thereon to be readily separated from unbound components during washing steps using a magnetic concentrator. A sample comprising the analyte is incubated with the first compound allowing the analyte to bind thereto, which followed by an incubation step with the second compound. In the present example the second compound is an antibody-DNA conjugate which binds the analyte at a different location than the first compound. The polynucleotide comprises a sequence which is capable of being bound and extended by human telomerase. Analyte-first compound-second compound complexes are then incubated with human telomerase in the presence of the appropriate nucleotide precursors including biotin-tagged dUTP. Biotin-tagged dUTP incorporated into the extended polynucleotide is then incubated with avidin labelled horseradish peroxidase (HRP). HRP activity is then used as an indicator of the amount of analyte in the sample by incubating the analyte-first compound-second compound-biotin dUTP-avidin-HRP complexes in the presence of hydrogen peroxide, luminol and iodophenol and detecting the luminescent signal in a luminometer.

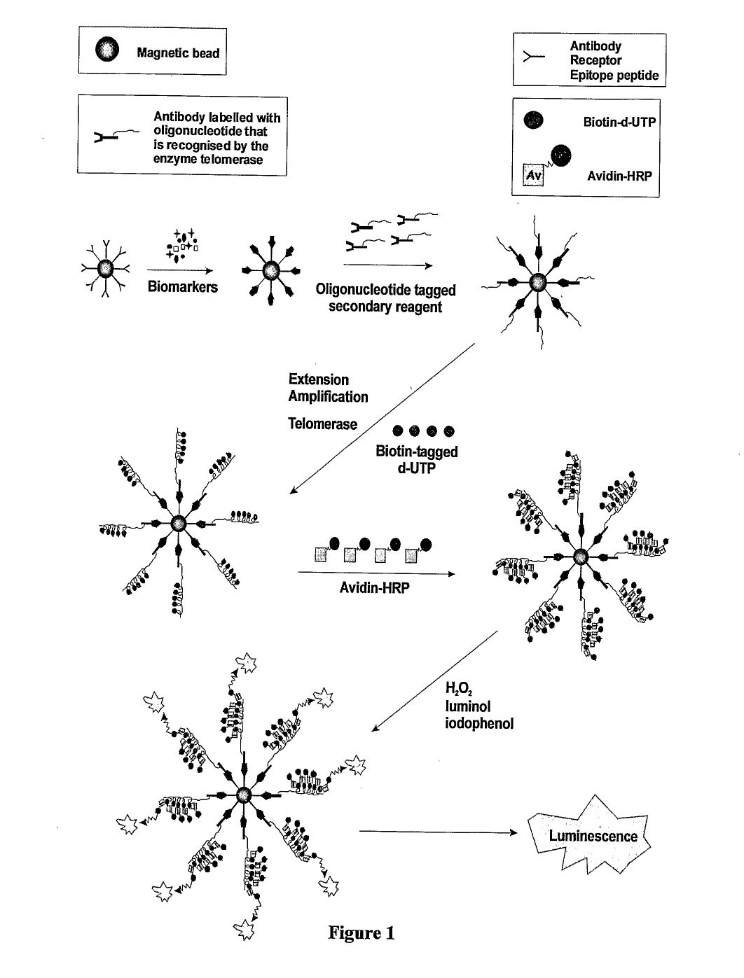

FIG. 2—BIAcore analysis of the simultaneous binding of NY-ESO-1 to two anti NY-ESO-1 antibodies (ES121 and E978).

FIG. 3—Bead-based sandwich ELISA using anti-NY-ESO-1 antibody coupled beads and NY-ESO-1 positive melanoma cell line lysate as the antigen indicate a linear response in direct relation to the number of melanoma cells.

FIG. 4—Bead-based sandwich ELISA using anti-EGFR antibody coupled beads and purified EGFR as the antigen indicate a linear response in direct relation to the concentration of recombinant protein.

FIG. 5—Separation of antibodies modified by the crosslinker from unmodified antibody on a Superdex 75 column (Amersham Bioscience) demonstrates that the coupling reaction has been successful using two different antibodies and that these reagent can be purified on the basis of apparent molecular size.

FIG. 6—1.5% Agarose gel following conjugation with reduced oligonucleotide. These data corroborate the observations reported in FIG. 5 whereby antibody bound to the oligonucleotide generates a strong band when stained with ethidium bromide.

FIG. 7—Separation profile of conjugated antibodies from excess oligonucleotide using size exclusion chromatography on a Superose 12 column (30/10) (Amersham Bioscience). These data support the prediction made in FIG. 5 that purification of oligo-bound antibodies can be achieved using standard chromatography.

FIG. 8—1.5% Agarose gel showing oligonucleotide conjugated antibodies following purification by size exclusion chromatography on a Superose 12 column (30/10) (Amersham Bioscience). Peak elution fractions 8, 9, 10 for each antibody are shown.

FIG. 9—SDS-PAGE (3-8% Tris-Acetate) of antibodies before conjugation (lanes 1 & 3) and purified conjugated antibodies (lanes 2 & 4) demonstrates that the oligo-bound antibody complex is of the appropriate and expected molecular mass.

FIG. 10—Mono Q elution profile of a conjugated antibody following Superose 12 chromatography. The peak heterogeneity suggests that multiple oligonucleotides might be coupled to antibodies providing further opportunities for reaction extensions and thus increase the detection capacity in the assay of the present invention.

FIG. 11—1.5% Agarose gel of oligonucleotide-conjugated antibodies following separation by ion exchange chromatography (MonoQ column). Independent confirmation that some of the oligo-conjugated antibodies have multiple nucleic acid moieties attached.

FIG. 12—Extension of oligonucleotide bound to antibody using telomerase. An increase luminescence indicates that there is an increase in the incorporation of biotinylated UTP in the presence of telomerase.

FIG. 13—Results (n=3) of assay for NY-ESO-1 detection by amplified luminescence.

FIG. 14—Detection of magnetic-bead bound oligonucleotides following signal amplification using terminal transferase.

FIG. 15—Bead-based assay demonstrating extension of the telomerase recognition sequence coupled to an antibody. Terminal transferase was used for the extension reaction with fluorescein-dUTP as a substrate. HI=heat inactivated.

FIG. 16—An amplified protein luminescence assay showing specific detection of the soluble EGFR (residues 1-621) by an LMH42-oligonucleotide conjugate in the presence of terminal transferase and fluorescein-dUTP.

FIG. 17—Bead-based telomerase assay showing the activity of the enzyme in crude and pre-cleared extracts derived from a bladder cancer cell line (LAR41). HI=heat inactivated.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

The “sample” refers to a material suspected of containing the analyte of interest. The sample can be used as obtained directly from the source or following at least one step to at least partially purify the analyte of interest from the sample obtained directly from the source. Such samples can include, for example, human, animal, plant, microorganism or man-made samples. The sample can be prepared in any convenient medium which does not interfere with the assay. Typically, the sample is an aqueous solution or biological fluid as described in more detail below. The sample can be derived from any source, such as a physiological fluid, including blood, serum, plasma, saliva, sputum, ocular lens fluid, sweat, faeces, urine, milk, ascites fluid, mucous, synovial fluid, peritoneal fluid, transdermal exudates, pharyngeal exudates, bronchoalveolar lavage, tracheal aspirations, cerebrospinal fluid, semen, cervical mucus, vaginal or urethral secretions, amniotic fluid, and the like. Herein, fluid homogenates of cellular tissues such as, for example, hair, skin and nail scrapings, meat extracts and skins of fruits and nuts are also considered biological fluids. Pretreatment may involve preparing plasma from blood, diluting viscous fluids, and the like. Methods of treatment can involve filtration, distillation, separation, concentration, inactivation of interfering components, and the addition of reagents. Besides physiological fluids, other samples can be used such as water, food products, soil extracts, and the like for the performance of industrial, environmental, or food production assays as well as diagnostic assays. In addition, a solid material suspected of containing the analyte can be used as the test sample once it is modified to form a liquid medium or to release the analyte. The selection and pretreatment of biological, industrial, and environmental samples prior to testing is well known in the art and need not be described further.

The term “analyte” refers to a substance to be detected or assayed by a method of the present invention. Typical analytes may include, but are not limited, to organic molecules, inorganic molecules, proteins, peptides, cells, microorganisms and fragments and products thereof, or any substance for which attachment sites, binding members or receptors (such as antibodies) can be developed.

As used herein, “nucleotide” refers to a base-sugar-phosphate combination. Nucleotides are monomeric units of a nucleic acid sequence (DNA and RNA). The term nucleotide includes deoxyribonucleoside triphosphates such as dATP, dCTP, dITP, dUTP, dGTP, dTTP, or derivatives thereof. Such derivatives include, for example, 7-deaza-dGTP and 7-deaza-dATP. According to the present invention, a “nucleotide” may be unlabeled or detectably labeled by well known techniques. However, at least one type of nucleotide used in the methods of the invention will be detectably labeled or conjugated to an appropriate hapten.

The term “hapten” refers to any molecule which can be linked to a nucleotide and incorporated into a polynucleotide by a polymerase. Furthermore, the hapten must be capable of binding at least one molecule (referred to generally herein as a “ligand” for the “hapten”) that is linked to a suitable detectable label such as those described herein. As the skilled addressee would be aware, the phrases “hapten” and “ligand” are merely used as convenient terms to define embodiments of the present invention. In particular, the hapten and ligand are members of a binding pair, however, it is often irrelevant which member of the binding pair is linked to, for example, the nucleotide. For instance, in one embodiment the hapten can be biotin and the ligand can be streptavidin, whereas as in another embodiment the hapten can be streptavidin and the ligand can be biotin. Useful haptens and ligands (“binding pairs”) for use in the methods of the invention are well known in the art.

### Polynucleotide Conjugates

The methods of the present invention require a conjugated compound comprising a polynucleotide and a molecule (for example a protein such as an antibody) that binds a target of interest. In many embodiments, the target of interest is the analyte which is being detected. However, the present invention also provides the use of a “universal” conjugated compound which is directed against a molecule (for example an antibody) that directly binds the analyte.

The polynucleotide may be DNA or RNA or a combination thereof. The polynucleotide may be single-stranded or double-stranded or a combination thereof.

As used herein, the term “polynucleotide” also refers DNA and/or RNA derivates that are still capable of being extended or act as a template for polynucleotide synthesis. An example of such derivates are peptide nucleic acids (PNA). Instead of having a ribose sugar backbone, PNAs typically have a backbone composed of repeating N-(2-aminoethyl)-glycine units linked by peptide bonds. The various pyrimidine and purine bases are linked to the backbone by methylene carbonyl bonds.

PNAs are less susceptible to degradation and form stronger duplexes with DNA than compared to DNA/DNA duplexes.

The polynucleotide is conjugated to the molecule that binds a target of interest by any technique known in the art. Examples include, but are not limited to, the use of biotin-avidin interaction, formation of disulfide bridges, amine coupling (see, for example, Hendrickson et al., 1995), thiol coupling (see, for example, Niemeyer et al., 2003), or aldehyde-hydrazine interaction (see, for example, Kozlov et al., 2004). Other coupling agents known to those in the art, include m-maleimidobenzoyl N-hydroxysuccinimide ester or related compounds, carbodiimides, such as, 1-ethyl-3-(3-diethylaminopropyl)carbodiimide (EDC), succinimidyl 4-(N-maleimidomethyl)cyclohexane-1-carboxylate (SMCC), and glutaraldehyde cross-linkers.

As the skilled person would appreciate, more than one polynucleotide can be conjugated to a single molecule (for example an antibody). This may increase the sensitivity of the assay.

For example, the polynucleotide can be conjugated to avidin, preferably streptavidin or neutravidin, and then linked to a biotinylated antibody. Further, for example, a method analogous to that described by Chu et al. (1986) can be used where biotin is attached to the 5′ terminus of the polynucleotide via a disulfide linker, and the biotinylated polynucleotide combined with avidin to form a polynucleotide-biotin-avidin adduct, which then could be conjugated to biotinylated antibodies. Other methods of attaching avidin to a polynucleotide are known to those in the art such as methods employing Protein A (Sano et al., 1992). molecule (for example a protein such as an antibody) that binds a target of interest

It will be appreciated by those skilled in the art that, in some cases, coupling of the polynucleotide to the molecule (for example a protein such as an antibody) that binds a target of interest may cause steric hindrance, resulting in reduced access to the polymerase. This can be circumvented by the use of a suitable linker such as those described previously in the literature including the linkers discussed in Kwon et al. (2004), Arora et al. (2002) and Ansari et al. (2001).

### Polymerases

In general, any polymerase capable of extending a polynucleotide, and/or synthesizing a complementary strand of a polynucleotide, can be used in the methods of the invention.

Examples of suitable DNA dependent DNA polymerases for use in the methods of the present invention include, but are not limited to, Tth DNA polymerase, Vent DNA polymerase, Pwo polymerase, DNA polymerase I Klenow fragment from bacteria such as E. coli, and T4 DNA polymerase.

Examples of suitable RNA dependent DNA polymerases for use in the methods of the present invention include, but are not limited to, AMV reverse transcriptase and M-MLV reverse transcriptase, SuperScript III and Tth polymerase.

Examples of suitable DNA dependent RNA polymerases for use in the methods of the present invention include, but are not limited to, T7 RNA polymerase, SP6 RNA polymerase and T3 RNA polymerase.

Examples of suitable RNA dependent RNA polymerases for use in the methods of the present invention include, but are not limited to, Qβ replicase, Hepatitis C RdRp, Vesicular Stomatitis Virus RdRp, Turnip yellow mosaic virus replicase and RNA bacteriophage phi 6 RNA-dependent RNA polymerase.

In a particularly preferred embodiment, the polymerase extends a single stranded polynucleotide or a single stranded overhang of a partially double stranded polynucleotide. Examples of such polymerases include, but are not limited to, poly(A)polymerase, T4 RNA ligase, telomerases and terminal transferases.

Telomerase can be isolated from immortal human cells for use in the methods of the invention. Telomerase may be purified by extraction in either hypotonic buffer or non-ionic detergent. It can also be purified by passing over a DEAE column and subsequent purification techniques. However, the telomerase may be obtained by merely lysing the appropriate cells without the need to perform any purification steps. The source of cells containing telomerase would be human tumor cell lines such as LIM1215 (Whitehead et al., 1985), Hela cells, HEK293 cells (Graham et al., 1977) and T-75 cells (van Bokhoven et al., 2001).

Telomerases can also be isolated from many other sources such as yeast or Tetrahymena (Bryan et al., 1998). Alternatively, telomerases can be produced using recombinant procedures well known to those skilled in the art.

Examples of suitable polynucleotide sequences which will be extended by human telomerase include, but are not limited to, TTAGGGTTAGGGTTAGGG (SEQ ID NO:1), TTTTTTAATCCGTCGAGCAGAGTT (SEQ ID NO:2), TTTTTTAATCCGTCGAGCAGAGTTAGGGTTAGGGTTAG (SEQ ID NO:3) and TTTTTTAATCCGTCGAGCAGAGTTAG (SEQ ID NO:4). As the skilled addressee would be aware, the above polynucleotides may be truncated or extended and still be useful for the methods of the invention.

Tetrahymena synthesizes a telomere repeat of 5′ TTGGGG 3′ (SEQ ID NO:5). The template on an encoding sequence is cloned and can be altered in the sequence to encode, the human telomere repeat 5′ TTAGGG 3′ (SEQ ID NO:6). The Tetrahymena enzyme may then be reconstituted with the altered RNA sequence to produce telomerase enzymes synthesizing the human telomeric sequence. This enzyme can be obtained in large quantities from Tetrahymena, purified and added to cells.

It is known in the art that telomerases are typically produced by cancerous cells. Thus, when telomerase (particularly a mammalian telomerase) is being used as an enzyme in the methods of the invention for the purposes of detecting a cancer analyte it is preferred that the analyte is separated from any telomerase that may be endogenously contained in the sample obtained from a subject.

Terminal transferase is a polymerase which catalyzes the repetitive addition of mononucleotides from dNTPs to the terminal 3′ OH of a polynucleotide substrate. The much preferred substrate for this enzyme is protruding 3′ ends, but it will also add nucleotides to blunt and 3′ recessed ends of DNA fragments. Cobalt is typically a necessary cofactor for activity of this enzyme, which in certain circumstances, may be added to the assay. Terminal transferase is a mammalian enzyme, expressed in lymphocytes. The enzyme can be purchased commercially and is usually produced by expression of the bovine gene in E. coli. An example of a commercial source is Promega (Madison, Wis., USA).

Poly(A) polymerase catalyzes the addition of adenosine to the 3′ end of RNA in a sequence-independent fashion. A consensus AAUAAA (SEQ ID NO:7) is typically used about 10-30 nucleotides 5′ of the polyA site and a GU rich and/or U rich element 3′ of the site. The AAUAAA signal is typically sufficient for polyadenylation initiation and extension if it is located at the appropriate distance from the end of a molecule. The skilled addressee can readily produce RNA molecules capable of being used as a substrate for poly(A) polymerase. As outlined herein, the enzyme can be used to append labeled ATP to the 3′ end of RNA molecules to generate labeled RNAs. Poly(A) polymerase can be produced recombinantly or obtained by purification from essentially any eukaryotic cell. Commercial sources for poly(A) polymerase useful for the methods of the invention include, Ambion Inc (Austin, Tex., USA), Invitrogen (Carlsbad, Calif., USA) and USB Corporation (Cleveland, Ohio, USA).

### Polynucleotide Synthesis and the Detection Thereof

In one embodiment, the conjugated polynucleotide is used as a template for the polymerase to synthesize a complementary strand. In another embodiment, the conjugated polynucleotide is extended by the action of the polymerase.

In one embodiment, polymerase activity is primed by annealing a suitable primer to a region of the conjugated polynucleotide. The primer can be any length or base composition, as long as it is capable of specifically annealing to a region of the polynucleotide. The extension is performed in the presence of one or more types of nucleotide triphosphates, and if desired, auxiliary binding proteins.

Incorporation of the dNTP is preferably determined by assaying for the presence of a hapten associated with an incorporated nucleotide(s). In a preferred embodiment, the synthesised polynucleotide strand is detected by measuring the presence of a biotin molecule linked to the specific dNTP. The presence of the biotin associated with the polynucleotide chain can be revealed via an enzyme-linked streptavidin molecule and, for example, a chemiluminescent substrate.

In one embodiment, hapten-labeled nucleotides (for example biotin, digoxigenin) are incorporated into the extending polynucleotide strand by the polymerase. An enzyme conjugated with a hapten-binding protein (ligand) is then added to label the polynucleotide. Chemiluminescent substrate for the enzyme (for example alkaline phosphatase, horseradish peroxidase, or β-galactosidase) is added to generate light (recorded by the detector).

Suitable enzymes for converting substrates into light include luciferases, for example, insect luciferases. Luciferases produce light as an end-product of catalysis. The best known light-emitting enzyme is that of the firefly, Photinus pyralis (Coleoptera) (see, for example, U.S. Pat. No. 5,618,722). In addition, a number of luciferase genes from the Jamaican click beetle, Pyroplorus plagiophihalamus (Coleoptera), have been cloned. Firefly luciferase catalyzes bioluminescence in the presence of luciferin, adenosine 5′-triphosphate (ATP), magnesium ions, and oxygen, resulting in a quantum yield of 0.88. Distinct luciferases can sometimes produce light of different wavelengths, which may enable simultaneous monitoring of light emissions at different wavelengths.

Luciferase can hydrolyze dATP directly with concomitant release of a photon. This results in a false positive signal because the hydrolysis occurs independent of incorporation of the dATP by polymerase activity. To avoid this problem, a dATP analog can be used which is incorporated into DNA, i.e., it is a substrate for a DNA polymerase, but is not a substrate for luciferase. One such analog is α-thio-dATP. Thus, use of α-thio-dATP avoids the spurious photon generation that can occur when dATP is hydrolyzed without being incorporated into a growing nucleic acid chain.

Examples of enzymes for which there are commercially available chemiluminescent substrates include β-galactosidase, alkaline phosphatase, neuraminidase, and horse-radish peroxidase.

Alkaline phosphatase is frequently conjugated to streptavidin, avidin, or antibodies to be used as secondary detection reagents. These detection reagents are widely used in a variety of applications including ELISAs, and Northern, Southern and Western blot techniques. Chromogenic substrates (such as BCIP, which yields a dark blue precipitate), fluorogenic phosphotase substrates, and chemiluminescent substrates are available. CDP-Star™ and CSPD™ (available from Applied Biosystems, Foster City, Calif., USA) chemiluminescent substrates for alkaline phosphatase enable the detection of alkaline phosphatase and alkaline phosphatase-labeled molecules with relative sensitivity, speed, and ease.

NA-Star™ chemiluminescent substrate (Applied Biosystems) enables sensitive detection of neuraminidase activity. This substrate is a highly sensitive replacement for the widely used fluorogenic substrate, methylumbelliferyl N-acetylneuraminic acid. 1,2-Dioxetane chemiluminescence substrates enable extremely sensitive detection of biomolecules by producing visible light that is detected with film or instrumentation. Chemiluminescence substrates emit visible light upon enzyme-induced decomposition, providing low background luminescence coupled with high intensity light output.

Chemiluminescent substrates are available for horse-radish peroxidase (HRP) from several manufacturers, including Alpha Diagnostic International, Inc. (San Antonio, Tex.) and Pierce Biotechnology Inc. (Rockford, Ill., USA). Alpha Diagnostic International's Nu-Glo substrate is provided as a stable two-component solution, and is a luminol-based solution. In the presence of hydrogen peroxide, HRP converts luminol to an excited state dianion that emits light on return to its ground state. The resulting signal can be measured by using a camera luminometer or X-ray films to provide a permanent record.

Luminescence may be detected and quantified using a variety of detection apparatuses, e.g., film, a photomultiplier tube, a CCD, CMOS, absorbance photometer, a luminometer, charge injection device (CID), or other solid state detector, as well as the apparatuses described herein. In one embodiment, the quantitation of the emitted photons is accomplished by the use of a CCD camera fitted with a fused fiber optic bundle. In another embodiment, the quantitation of the emitted photons is accomplished by the use of a CCD camera fitted with a microchannel plate intensifier. A back-thinned CCD can be used to increase sensitivity. CCD detectors are described by Bronks et al. (1995).

An exemplary CCD system is a Spectral Instruments, Inc. (Tucson, Ariz., USA) Series 600 4-port camera with a Lockheed-Martin LM485 CCD chip and a 1-1 fiber optic connector (bundle) with 6-8 μm individual fiber diameters. This system has greater than 16 million pixels and has a quantum efficiency ranging from 10% to >40%. Thus, depending on wavelength, as much as 40% of the photons imaged onto the CCD sensor are converted to detectable electrons.

In other embodiments, a fluorescent moiety can be used as a label and the detection of a reaction event can be carried out using a confocal scanning microscope. Additionally, scanning tunneling microscopy and atomic force microscopy can be used.

Other examples of substrates (labels) that can be detectable by emitted photons can be utilized in the methods of the invention. Reaction of the acridan substrate with an enzyme results in an excited intermediate that can give off light. For example, the reaction can be between the Pierce Lumi-Phos WB substrate and alkaline phosphatase, though the enzyme used can vary depending on the cleavable moiety substituted onto the acridan molecule. Reaction of the luminol substrate with peroxidase results in an unstable intermediate that emits light and is converted into the 3-aminophtalate dianion. This is the reaction that occurs in the Pierce SuperSignal™ ELISA Femto Maximum Sensitivity Substrate. Furthermore, reaction of the 1,2-dioxetane substrate with an enzyme results in an unstable intermediate that breaks apart to yield two product molecules, adamantanone and a chemically excited fluorophor, which can then give off light. For example, the reaction can be between Lumigen PPD and alkaline phosphatase. The enzyme used can vary depending on the cleavable moiety substituted onto the 1,2-dioxetane-based substrate.

For most applications it is desirable to wash away unincorporated reagents, for example, unincorporated dNTPs, with a wash buffer. Any wash buffer which does not interfere with the formation/stability of the complexes, and/or the detection thereof, can be used. Such wash buffers are well known to those skilled in the art.

### Solid Supports

Typically, at least one of the reagents used in the methods of the invention will need to be attached to a suitable solid support. In a particularly preferred embodiment, the first compound is attached to the solid support. However, in certain instances an analyte may serve as the capture reagent by being absorbed directly by nonspecific interaction with the support, as in, for example, the hydrophobic interactions between proteins and polystyrene.

Suitable solid-phase supports for use in the methods of the invention are common and well known in the art. A variety of possible supports are contemplated. For example suitable immobilization supports include but are not limited to synthetic polymer supports, such as polystyrene, polypropylene, polyglycidylmethacrylate, substituted polystyrene (e.g., aminated or carboxylated polystyrene; polyacrylamides; polyamides; polyvinylchlorides, etc.); glass, gold, agarose, nitrocellulose, and nylon. These materials may be used as films, microtiter plate, wells, beads, slides, particles, pins, pegs, test tubes, membranes or biosensor chips. Alternatively, the supports comprise magnetic and non-magnetic particles. Preferred magnetic particles are magnetic beads such as those available from Dynal Biotech (Oslo, Norway), Polymer Labs Lodestar Beads (Shropshire, UK) or Millipore CPG beads (Millipore, Lane Cove, Australia).

Many procedures and linker molecules for attachment of various molecules to various metal, glass, plastic etc., substrates are well known to those of skill in the art (see, for example, EP 188,256; U.S. Pat. No. 4,671,958, U.S. Pat. No. 4,659,839, U.S. Pat. No. 4,414,148, U.S. Pat. No. 4,699,784; U.S. Pat. No. 4,680,338; U.S. Pat. No. 4,569,789; and U.S. Pat. No. 4,589,071, as well as H. Weetall, Immobilized Enzymes, Antigens, Antibodies and Peptides, Marcell Dekker, Inc., New York (1975)). For example a “linker” can be used to attach an appropriate molecule to a solid support. Suitable linkers are well known to those of skill in the art and include, but are not limited to, straight or branched-chain carbon linkers, heterocyclic carbon linkers, or peptide linkers.

A bifunctional linker having one functional group reactive with a group on the surface, and another group reactive with the desired molecule (for example the first compound) may be used to form the required conjugate. Alternatively, derivatization may involve chemical treatment. For example, a silica or glass substrate can be silanized to create functional group. Similarly, a protein or glycoprotein, can be derivatized, e.g., by glycol cleavage of a sugar moiety attached to the protein antibody with periodate to generate free aldehyde groups. The free aldehyde groups on the antibody or protein or glycoprotein may be reacted with free amine or hydrazine groups on the surface to bind the binding partner thereto (see U.S. Pat. No. 4,671,958). Procedures for generation of free sulfhydryl groups on polypeptide, such as antibodies or antibody fragments, are also known (see U.S. Pat. No. 4,659,839).

An array can be used to carry out separate parallel common reactions in an aqueous environment. The array can have a substrate having at least 1,000 discrete reaction chambers containing a starting material that is capable of reacting with a reagent, each of the reaction chambers being dimensioned such that when one or more fluids containing at least one reagent is delivered into each reaction chamber, the diffusion time for the reagent to diffuse out of the well exceeds the time required for the starting material to react with the reagent to form a product. The reaction chambers can be formed by generating a plurality of cavities on the substrate. The plurality of cavities can be formed in the substrate via etching, molding or micromachining. The cavities can have a planar bottom or a concave bottom. In a preferred embodiment, the substrate is a fiber optic bundle. In an additional embodiment, the reaction chambers are formed by generating discrete patches on a planar surface. The patches can have a different surface chemistry than the surrounding planar surface.

### Antibodies

For the purposes of this invention, the term “antibody”, unless specified to the contrary, includes fragments of whole antibodies which retain their binding activity for a target analyte. Such fragments include Fv, F(ab′), F(ab′)₂ and dAb fragments, as well as single chain antibodies (scFv). Furthermore, the antibodies and fragments thereof may be humanised antibodies, for example as described in EP-A-239400.

Antibodies useful for the methods of the invention may be monoclonal or polyclonal. However, to reduce any problems with background signals it is preferred that the antibody(ies) is/are monoclonal.

The term “binds specifically” refers to the ability of the antibody to bind to a particular analyte but not other molecules in the sample and/or non-target analytes.

If polyclonal antibodies are desired, a selected mammal (e.g., mouse, rabbit, goat, horse, etc.) is immunised with the analyte of interest—such as an immunogenic polypeptide. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art.

Monoclonal antibodies directed against an analyte of interest can also be readily produced by one skilled in the art. The general methodology for making monoclonal antibodies by hybridomas is well known. Immortal antibody-producing cell lines can be created by cell fusion, and also by other techniques such as direct transformation of B-lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is also well known in the art.

### Uses

Suitable analytes which can be detected using the methods of the invention include organic and inorganic molecules, including biomolecules. In a preferred embodiment, the analyte may be an environmental pollutant (including pesticides, insecticides, toxins, etc.); a chemical (including solvents, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eucaryotic cells, including mammalian tumor cells); viruses (including retroviruses, herpesviruses, adenoviruses, lentiviruses, etc.); agents that can be used for bioterrorism (such as anthrax) and spores; etc. Particularly preferred analytes are environmental pollutants; nucleic acids; proteins (including enzymes, antibodies, antigens, growth factors, cytokines, etc); therapeutic and abused drugs; cells; and viruses.

Particularly preferred target analytes include proteins and nucleic acids. “Protein” as used herein includes proteins, polypeptides, and peptides. The protein may be made up of naturally occurring amino acids and peptide bonds, or synthetic peptidomimetic structures. The side chains may be in either the (R) or the (S) configuration.

The analyte to be detected includes hormones such as growth hormone, insulin, adrenocorticotropic hormone (ACTH), thyroid-stimulating hormone (TSH), luteinizing hormone (LH) and gut hormone, e.g., glicentin; growth factors such as epidermal growth factor (EGF), vascular endothelial growth factors such as VEGF, VEGF-B, VEGF—C, VEGF-D and VEGF-E, nerve growth factor (NGF), platelet-derived growth factor (PDGF) and related molecules, fibroblast growth factor (FGF), insulin-like growth factor (IGF) and hepatosite growth factor; bacterial toxins; bacterial metabolites and antibodies thereto; exosporium components; virus capsid components; enzymes such as alkaline phosphatase (ALP), glutamate-oxaloacetate transaminase (GOT), glutamate-pyruvate transaminase (GPT), lactate dehydrogenase (LDH), blood clotting factor; lipoproteins such as very low-density lipoprotein (VLDL), high-density lipoprotein (HDL) and low-density lipoprotein (LDL); receptors such as hormone receptors, e.g., insulin receptor, growth hormone receptor, EGF receptor and nerve receptor, e.g., acetylcholine receptor; cancer markers such as those described below; cell surface antigens such as histocompatibility antigen; autoantibodies; c-reactive proteins; and other physiological active substances such as peptide enzyme inhibitors, e.g., macroglobulin; and complements such as carrier protein, IGF-binding protein and transferrin.

The present invention can be used to detect the presence of a tumour antigen (analyte) in a sample. Examples of tumour antigens which can be detected using the methods of the invention include, but are not limited to, for AFP (marker for hepatocellular carcinoma and germ-cell tumours), CA 15-3 (marker for numerous cancers including breast cancer), CA 19-9 (marker for numerous cancers including pancreatic cancer and biliary tract tumours), CA 125 (marker for various cancers including ovarian cancer), calcitonin (marker for various tumours including thyroid medullary carcinoma), catecholamines and metabolites (phaeochromoctoma), CEA (marker for various cancers including colorectal cancers and other gastrointestinal cancers), epithelial growth factor (EGF) and/or epithelial growth factor receptor (EGFR) (both associated with a range of epithelial cancers including colon cancer), A33 colonic epithelial antigen (colon cancer), hCG/beta hCG (marker for various cancers including germ-cell tumours and choriocarcinomas), 5HIAA in urine (carcinoid syndrome), PSA (prostate cancer), sertonin (carcinoid syndrome), thyroglobulin (thyroid carcinoma), and the CT antigens (Scanlan et al., 2002) such as NY-ESO-1 (marker of oesophageal and bladder cancer and melanoma), LAGE, MAGE (associated with many liver cancers and melanomas), GAGE (hepatocarcinoma), SSX2 (sarcoma) differentiation antigens (such as Melan A/MART1, GP100 and tyrosinase), mutational antigens (such as CDK4, β-catenin), amplification antigens (such as P53 and Her2), and splice variant antigens (such as ING1).

The present invention can also be used to detect the presence of a microorganism, and/or analyte produced thereby, in a sample. The target may be, but not limited to, a virus, bacteria, fungi or protozoa. Specific non-limiting examples of microorganisms to which the invention can be suitably applied include bacteria such as Mycobacteria tuberculosis, Rickettsia rickettsii, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffli, Mycoplasma fermentans, Ehrlichia sp., Haemophilus influenzae, Neisseria meningitidis, Streptococcus pneumonia, S. agalactiae, and Listeria monocytogenes; viruses such as Human Immunodeficiency Virus Type 1 (HIV-1), Human T-Cell Lymphotrophic Virus Type 1 (HTLV-1), Hepatitis B Virus (HBV), Hepatitis C Virus (HCV), Herpes Simplex, Herpesvirus 6, Herpesvirus 7, Epstein-Barr Virus, Cytomegalo-virus, Varicella-Zoster Virus, JC Virus, Parvovirus B19, Influenza A, B and C, Rotavirus, Human Adenovirus, Rubella Virus, Human Enteroviruses, Genital Human Papillomavirus (HPV), and Hantavirus; fungi such as Cryptococcus neoformans, Pneumocystis carinii, Histoplasma capsulatum, Blastomyces dermatitidis, Coccidioides immitis, and Trichophyton rubrum; and protozoa such as Trypanosoma cruzi, Leishmania sp., Plasmodium sp., Entamoeba histolytica, Babesia microti, Giardia lamblia, Cyclospora sp. and Eimeria sp. The method of the invention may also be used for Cryptosporidium sp. oocyst detection; for identification of bacterial toxins, such as the toxin genes from Vibrio cholerae 01, enterotoxigenic Escherichia coli, Shigella sp., enteroinvasive E. coli, Helicobacter pylori, toxigenic Clostridium difficile, Staphylococcus aureus, and Streptococcus pyogenes exotoxins.

### Kits

At least some of the materials and reagents required for the disclosed detection methods may be assembled together in a kit. The kits of the present disclosure generally will include at least the polymerase and nucleotides necessary to carry out the claimed methods. In a preferred embodiment, the kit comprises a protein-DNA conjugate wherein the DNA comprises a sequence which can bind, and be extended by, a telomerase. In another preferred embodiment, the kit will also contain directions for detecting an analyte in a sample. The kit may also comprise means for detecting the activity of the polymerase.

The kit may also contain control samples provided at varying concentrations to enable the user to construct an appropriate standard curve. For example, a kit for the detection of NY-ESO-1 may comprise recombinant NY-ESO-1 provided at concentrations of 25, 50, 100, 250 and 500 ng/ml.

In each case, the kits will preferably have distinct containers for each individual reagent and polymerase. Each biological agent will generally be suitably aliquoted in their respective containers. The container means of the kits will generally include at least one vial or test tube. Flasks, bottles, and other container means into which the reagents are placed and aliquoted are also possible. The individual containers of the kit will preferably be maintained in close confinement for commercial sale.

### EXAMPLES

### Example 1

### Simultaneous Binding of NY-ESO-1 to Two Anti-NY-ESO-1 Antibodies (ES121 and E978)

Mouse-antihuman-NY-ESO-1 mAb ES121 (Jungbluth et al., 2001) was immobilised onto a CM5 BIACore surface plasmon resonance (SPR) biosensor surface via amine coupling chemistry. The recombinant antigen NY-ESO-1 (150 μg/ml in 40 mM Urea/DDW) (Murphy et al., 2005) was injected over the ES121 immobilised surface at a flow rate of 5 μl/min. An injection of antibody ES121 at a flow rate of 5 μl/min was performed to ensure no non-specific binding and then mouse-antihuman-NY-ESO-1 mAb E978 (Jungbluth et al, 2001) (Invitrogen Carlsbad, Calif., USA) was injected over the surface.

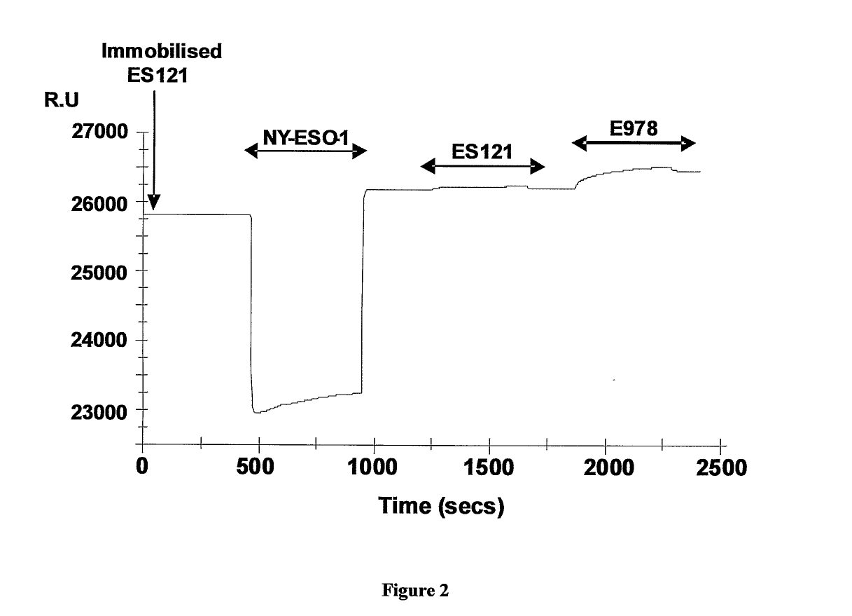

Results show that the antigen NY-ESO-1 binds with relatively high affinity to the immobilised ES121 mAb surface and that when another injection of ES121 mAb was injected over the antibody-antigen surface then little non-specific binding was seen (FIG. 2). The final injection of mAb E978 shows that it can recognise and bind to NY-ESO-1 captured on the surface with high affinity and specificity. This experiment has been performed in the reverse order of antibody binding with similar results (data not shown).

### Example 2

### Primary Antibody Coupling and Characterisation

M270 Carboxylic Acid Dynal® beads were coupled to anti-NY-ESO-1 antibody (E978) or anti-EGFR antibody. The respective antibodies (25 mM MES pH 5) were covalently coupled to the M270 carboxylic acid beads via amine coupling chemistry using 1-ethyl-3-(3-dimethylaminopropyl)carbodiimide (EDC)/N-hydroxysuccinimide (NHS) reagents in accordance with the manufacturer's instructions. Coupling efficiency was monitored by Size Exclusion Chromatography (SEC) using a Superose 12 column (10/30).

To determine successful mAb coupling, and to confirm that the mAb was still biologically active, an automated bead-based sandwich ELISA assay was developed. The antibody coupled to the magnetic beads (300 μg/ml of coupled beads) was incubated with its respective antigen (either cell lysate or purified protein), this complex was then incubated with an alternative anti-NY-ESO-1 or anti-EGFR antibody (2 μg/ml in PBS/3% BSA buffer) which had been biotinylated. Biotinylation was performed as per ECL Protein Biotinylation Module Kit (Amersham Bioscience). For detection it was then incubated with streptavidin-HRP (1 μg/ml KPL) which is the substrate for the final chemiluminescence read-out. Chemiluminescence was produced upon the addition of 50 μl of Luminol and 50 μl of Peroxide (Supersignal ELISA Femto substrate, Pierce).

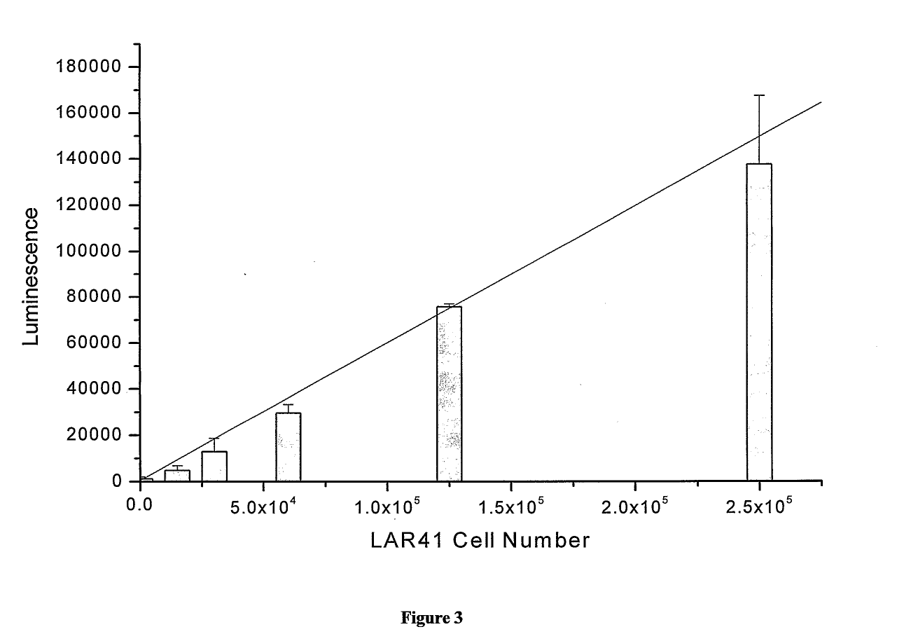

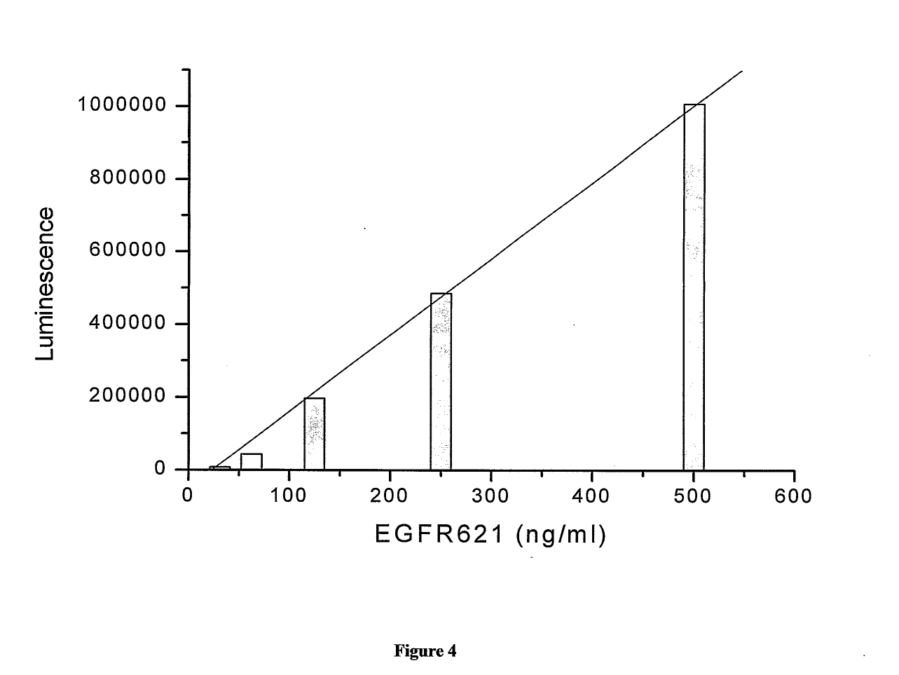

Results indicated that the coupling to the magnetic beads was successful for both examples and that the antibody was coupled in an orientation allowing binding of the respective antigen (FIGS. 3 and 4).

### Example 3

### Oligonucleotide-Antibody Coupling

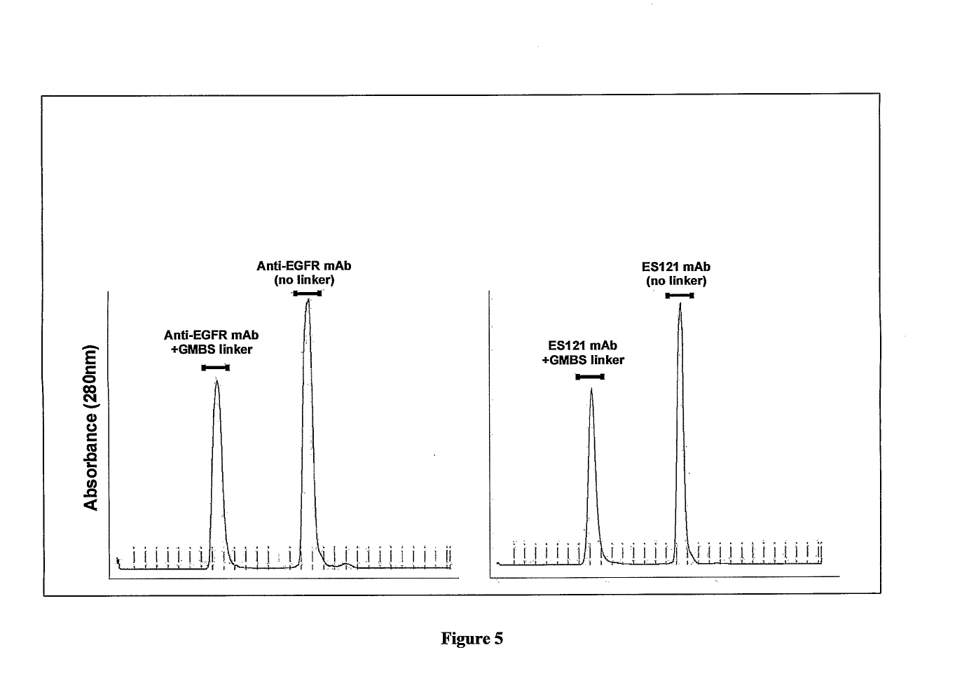

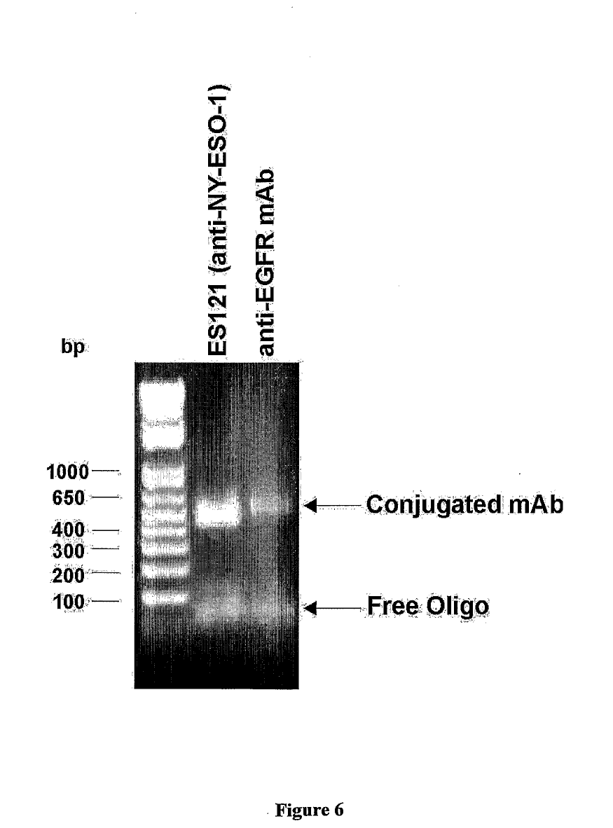

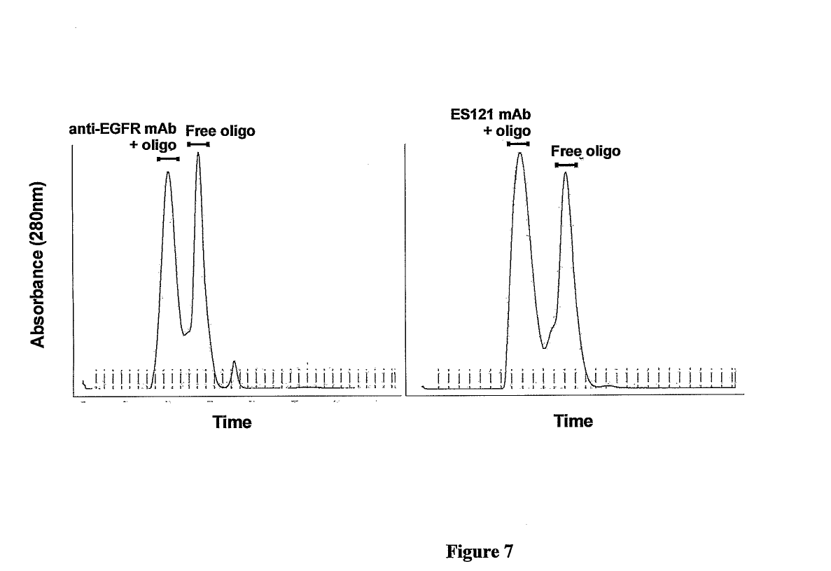

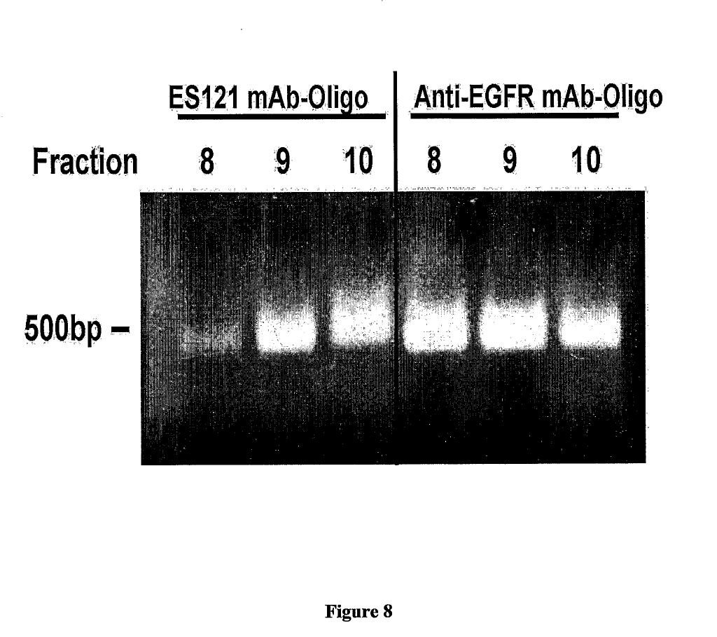

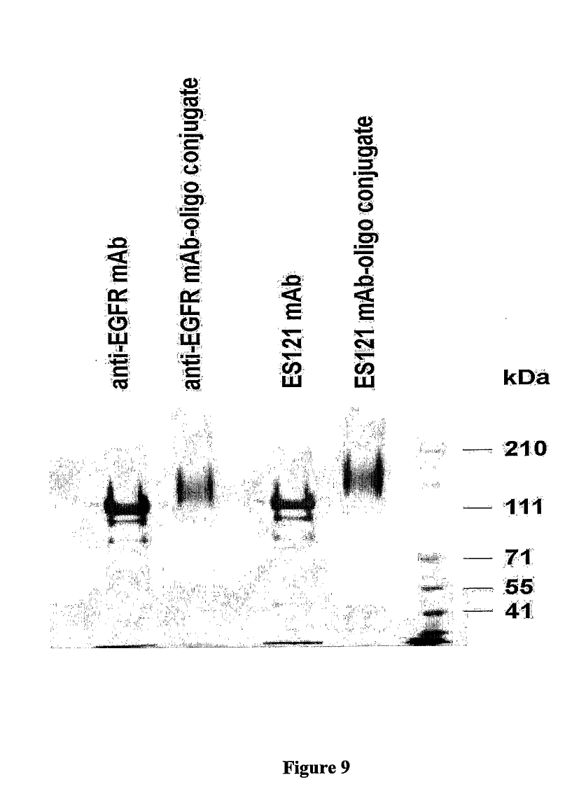

Two antibodies (anti-NY-ESO-1 (ES121) and anti-EGFR) were successfully conjugated with the telomerase recognition oligonucleotide (5′-TTTTTTAATCCGTCGAGCAGAGTTAGGGTTAGGGTTAG-3′-SEQ ID NO:3) which included a 5′ thiol modification to allow coupling via thiol coupling chemistry using the NHS-Esters-Maleimide crosslinker N-[γ-maleimidobutyryl-oxy]sulfosuccinimide ester (sulfo-GMBS, Pierce) (Schweitzer et al., 2000). The coupling and purity were monitored using HPLC, SDS-PAGE gel electrophoresis and TBE-agarose electrophoresis (FIGS. 5 to 9). Quantitation and stoichiometry was obtained spectrophotometrically using the BCA (Pierce) and Oligreen (Pierce) assays (see below). Absolute protein concentration was obtained by quantitative amino acid analysis.

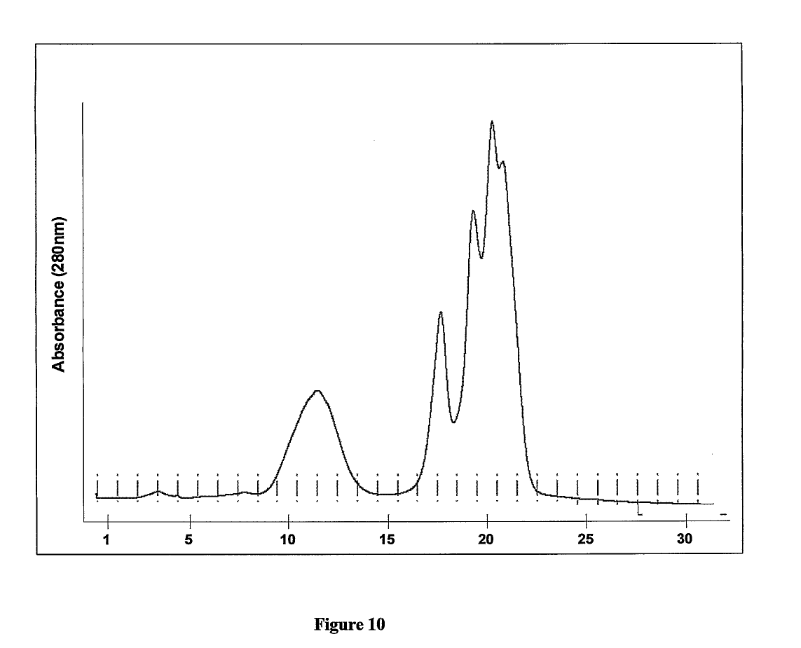

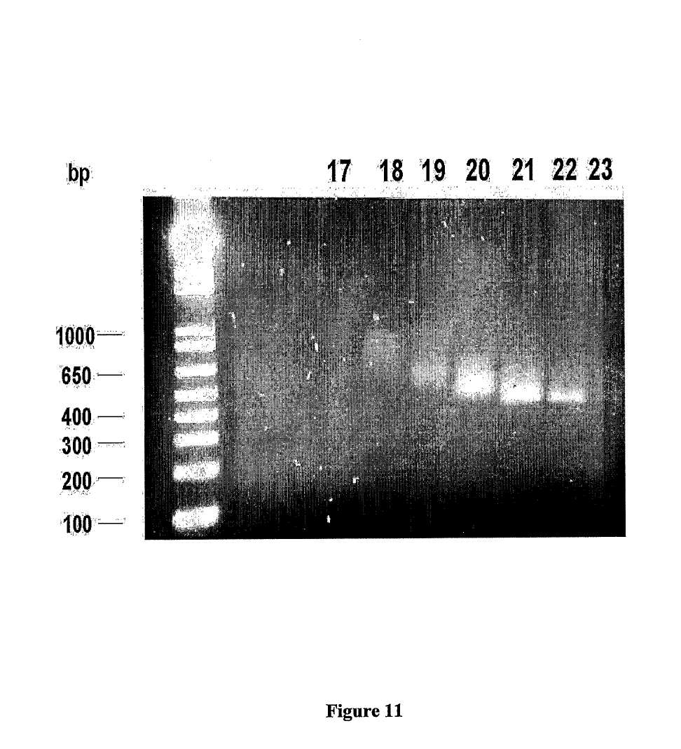

Further analysis of heterogeneity due to the coupling of multiple tags via lysine residues was achieved using Mono-Q chromatography (FIG. 10). The peaks recovered over fractions 17-22 were analysed by SDS-PAGE (data not shown) and 1.5% Agarose gels to show the different oligonucleotide-antibody isoforms produced (FIG. 11).

### Example 4

### Oligonucleotide-Antibody Characterisation

The oligonucleotide-antibodies described in Example 3 were analysed in a bead-based extension assay to ensure the oligonucleotide-antibodies were still biologically active and capable of binding NY-ESO-1 or EGFR respectively: this assay also indicates that the oligonucleotide recognition sequence can be extended by enzyme (telomerase).

Dynal® M270 amine magnetic beads were coupled with a high affinity form of the soluble EGFR via amine coupling chemistry (coupling protocol followed as per Dynal package insert). This assay was automated in the Kingfisher particle processor. 6 μL per well of EGFR coupled beads were blocked with blocking buffer (PBS/1% BSA) and then incubated for 30 minutes at RT with oligonucleotide-anti-EGFR antibody or for the unamplified assay anti-EGFR antibody-biotin. Biotinylation was performed as per ECL Protein Biotinylation Module Kit (Amersham Bioscience). The samples were then incubated with the reaction mix containing 20 mM Tris-HCl, 1.5 mM MgCl₂, 63 mM KCl, 1 mM EGTA, 1 mM EDTA, 150 mm NaCl, 0.005% Tween 20, 12.5 μM Biotin-21-dUTP, 18.75 μM dAdG, 1 μl telomerase and LIM1215 lysate from 10⁶ cells (Ludwig Institute Melbourne cell line 1215—Whitehead et al., 1985) for amplification. Heat inactivated telomerase (95° C. for 20 minutes) was included as a control. Following the amplification step, a number of washes were performed and the samples were then incubated with 0.5 μg/ml streptavidin-HRP for 30 minutes at RT. Following this incubation, the samples were washed with working buffer (0.1M Tris, 0.1M KCl, pH7.4) before being resuspended in a final volume of 50 μL of working buffer. The samples were then transferred to a white luminescence 96 well plate and read for luminescence in a BMG Fluorostar luminometer upon the addition of 50 μL Luminol and 50 μL Peroxide (Supersignal ELISA Femto substrate, Pierce).

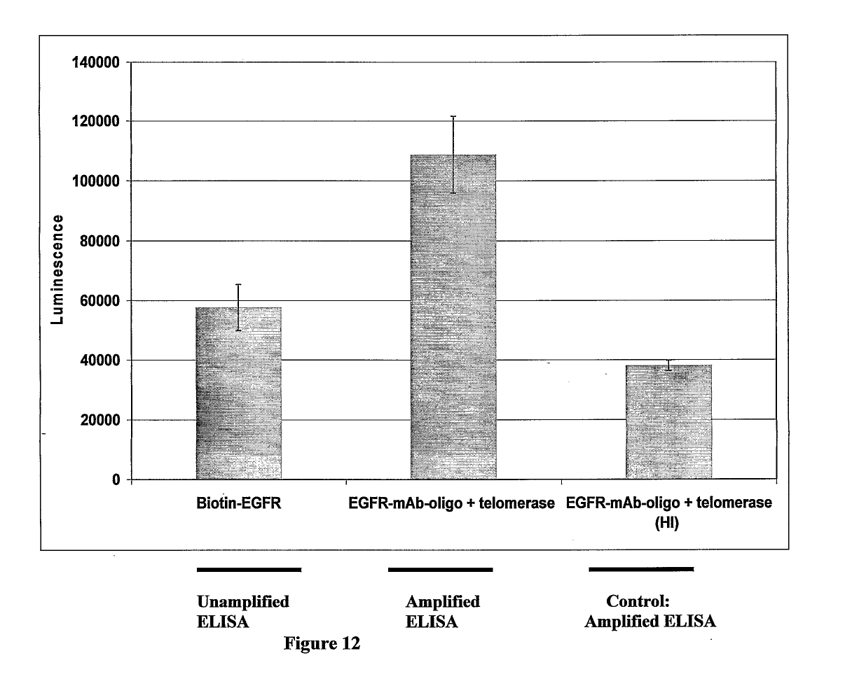

Results (n=2) indicate that the signal from the amplified assay is significantly higher than the heat inactivated control and the unamplified ELISA signal (FIG. 12).

### Example 5

### Oligonucleotide-Antibody Quantitation

The oligonucleotide content was quantitated by the Oligreen (Pierce) assay which is a fluorescent based assay that detects ssDNA. Quantitation of the oligonucleotide-antibodies is based on a standard curve generated with the unmodified oligonucleotide.

The antibody concentration was determined by a standard BCA assay, this allows for accurate estimation of protein content without the interference of the signal contributing to the oligonucleotide. The absorbance is read at 550 nm.

### Example 6

### Detection of NY-ESO-1 Using Telomerase

The magnetic beads coupled to E978 mAb (180 μg) were incubated with a NY-ESO-1 positive cytosolic extract (0.5×10⁷ cells for 60 minute at RT) isolated from the Melanoma cell line LAR41. The LAR (Ludwig Austin Repatriation) series of melanoma cell lines were derived at the Ludwig Institute from patients' biopsy samples, and consent was obtained from each patient before establishment.

Following binding of the antigen the beads were incubated with the ES121 mAb (16 μg/ml for 60 minutes at RT) which was tagged with an oligonucleotide recognition sequence for telomerase (5′-TTTTTTAATCCGTCGAGCAGAGTTAGGGTTAGGGTTAG-3′-SEQ ID NO:3) which included a 5′ thiol modification to allow coupling via thiol coupling chemistry using the NHS-Esters-Maleimide crosslinker N-[γ-maleimidobutyryl-oxy]sulfosuccinimide ester (GMBS) (as described above).

Telomerase enzyme isolated from HE 93 (Human embryonic kidney) cells was used to extend the oligonucleotide (60 minutes at 37° C.) in the presence of a Reaction mix buffer (20 mM Tris-HCl, 1.5 mM MgCl2, 63 mM KCl, 1 mM EGTA, 1 mM EDTA, 150 mm NaCl, 0.005% Tween 20, 12.5 μM Biotin-21-dUTP, 18.75 μM dAdG). This step adds a number of biotin residues along the DNA sequence as the enzyme extends it. The complex was then incubated with streptavidin-HRP (0.5 μg/ml for 30 minutes at RT). Following this incubation, the samples were washed with working buffer (0.1M Tris, 0.1M KCl, pH7.4) before being resuspended in a final volume of 50 μL of working buffer. The samples were then transferred to a white luminescence 96 well plate and read for luminescence in a BMG Fluorostar luminometer upon the addition of 50 μL Luminol and 50 μL Peroxide (Supersignal ELISA Femto substrate, Pierce).

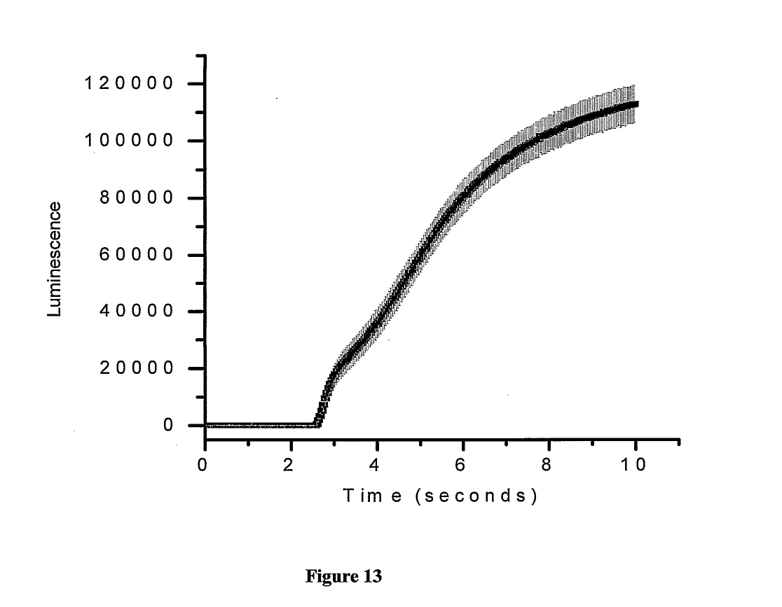

The assay was assembled using a Kingfisher particle processor for automation. Results (n=3) indicated a strong luminescence signal upon the addition of 50 μL luminol and 50 μL peroxide (Pierce) (FIG. 13).

### Example 7

### Extension of Oligonucleotide Template Bound to a Magnetic Bead Using Terminal Transferase

Magnetic dynal beads were coupled with a single stranded DNA oligonucleotide (TTTTTTAATCCGTCGAGCAGAGTTAGGGTTAGGGTTAG-SEQ ID NO:3). In this case it was the same sequence as used by the telomerase enzyme as terminal transferase does not preferentially extend a specific sequence like telomerase. Extension conditions were modified for this enzyme, the elongation buffer used was 50 mM Potassium Acetate, 20 mM Tris Acetate, 10 mM Magnesium Acetate, 0.25 mM Cobalt Chloride, 12.5 μM Biotin-21-dUTP, 18.75 μM dAdG, 10U terminal transferase, (pH 7.9). Terminal transferase was added to each well of 10 μL Oligo-coupled magnetic beads (30 mg/ml) with 100 μL of elongation buffer for 30 minutes at 37° C. The extended magnetic beads were then washed in working buffer (0.1M Tris, 0.1M Potassium Chloride) three times. The beads were then incubated with 0.5 μg/ml of Streptavidin-HRP for 30 minutes at room temperature and followed with three working buffer washes before being resuspended in a final volume of 50 μL of working buffer. The samples were then transferred to a white luminescence 96 well plate and luminescence read in a BMG Fluorostar luminometer following addition of 50 μL Luminol and 50 μL Peroxide (Pierce). Experiment was performed in duplicate, including a heat inactivated control where the terminal transferase was inactivated by heating at 95° C. for 20 minutes.

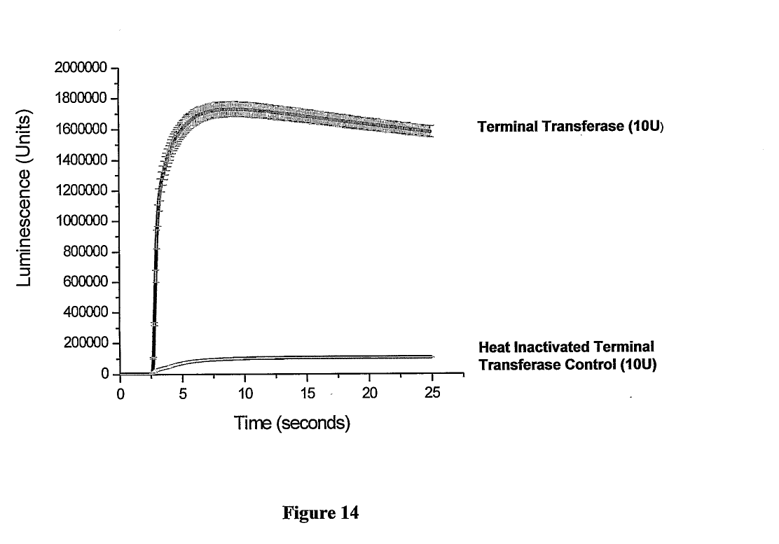

Results show a luminescence signal significantly higher than the heat inactivated control indicating that telomerase can be substituted for terminal transferase in this method (FIG. 14).

### Example 8

### Extension of Oligonucleotide Template Bound to an Antibody Using Terminal Transferase

Tosyl-activated Dynal Beads (15 mg) are coupled to 300 μg 501FC (the ligand-binding domain of the EGF receptor linked to an antibody Fc region) by overnight incubation at 37° C. in 0.1M borate; pH 9.6. After coupling the beads are washed well and unreacted groups capped by incubation with 0.2M Tris/0.1% BSA for 4 hours at 37° C. The beads were then washed and stored in PBS/0.1% BSA.

The anti-EGFR antibody LMH42 is functionalised with the target nucleotide containing the recognition sequence for telomerase (TTTTTTAATCCGTCGAGCAGAGTTAGGGTTAGGGTTAG) (SEQ ID NO:3) using hydrazine chemistry as described previously (Kozlov et al., 2004). The resulting conjugate is purified from residual reactants using size exclusion HPLC on a Superose 12 10/300 column (GE Amersham) using PBS buffer at a flow rate of 1 ml/min and a column temperature of 25° C.

A 10 μL volume of 501FC-conjugated beads are incubated in the presence of the LMH42 antibody functionalised with the telomerase recognition sequence for 60 minutes at room temperature followed by extensive washing. Bead-501FC-antibody complexes were subsequently treated with 1% BSA to reduce non-specific background signals. After further washing, complexes were incubated for 60 minutes at 37° C. in the presence of 50 μL reaction buffer (50 mM potassium acetate, 20 mM Tris-acetate, 10 mM magnesium acetate, 0.25 mM cobalt chloride, 12.5 μM fluorescein-dUTP, 18.75 μM dAdG; pH 7.9) and 0.5 μl of terminal transferase (10U of enzyme). The extended magnetic beads were washed three times in working buffer (0.1M Tris, 0.1M potassium chloride) and then incubated with 1 μg/ml of anti-fluorescein-HRP for 30 minutes at room temperature. Following this, the beads were washed three times with working buffer before being resuspended in a final volume of 50 μL of working buffer. The samples were then transferred to a white luminescence 96 well plate and luminescence read in a BMG Fluorostar luminometer following addition of 50 μL Luminol and 50 μL Peroxide (Pierce). Treatments were performed in duplicate, including a heat inactivated control where the terminal transferase was inactivated by heating at 95° C. for 30 minutes.

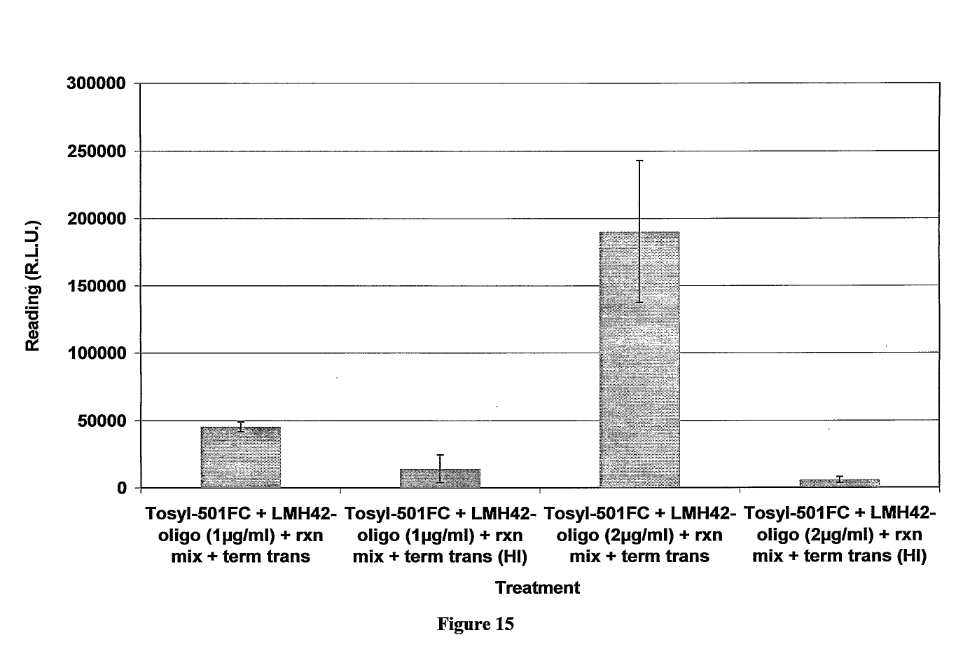

The results show that (FIG. 15) the terminal transferase is able to extend the oligonucleotide substrate and incorporate labelled nucleotides. Hence, terminal transferase is clearly useful for the methods of the invention.

### Example 9

### Specific Detection of EGFR Antigen Using Terminal Transferase and Fluorescein-dUTP

Tosyl-activated Dynal Beads (15 mg) were coupled to 300 μg LMH41 anti-EGFR mAb by overnight incubation at 37° C. in 0.1M borate buffer; pH 9.6. After coupling the beads are washed extensively and unreacted groups capped by incubation with 0.2M Tris/0.1% BSA for 4 hours at 37° C. The beads are then washed and stored in PBS/0.1% BSA.

The anti-EGFR antibody LMH42 was functionalised with the target nucleotide containing the recognition sequence for telomerase (TTTTTTAATCCGTCGAGCAGAGTTAGGGTTAGGGTTAG) (SEQ ID NO:3) using hydrazine chemistry as described previously (Kozlov et al., 2004). The resulting conjugate is purified from residual reactants using size exclusion HPLC on a Superose 12 10/300 column (GE Amersham) using PBS buffer at a flow rate of 1 ml/min and a column temperature of 25° C.

A 10 μL volume of LMH41-conjugated beads are incubated in the presence of different concentrations of EGFR antigen (sEGFR 1-621, Domagala et al., 2000) for 60 minutes at room temperature. Following a number of wash steps, the antigen-antibody-bead complexes were incubated with the LMH42 antibody functionalised with the telomerase recognition sequence for 60 minutes at room temperature followed by extensive washing. Bead complexes were subsequently washed and then incubated for 60 minutes at 37° C. in the presence of 50 μL reaction buffer (50 mM potassium acetate, 20 mM Tris-acetate, 10 mM magnesium acetate, 0.25 mM cobalt chloride, 12.5 μM fluorescein-dUTP, 18.75 μM dAdG; pH 7.9) and 0.5 μl of terminal transferase (10U of enzyme). The extended magnetic beads were washed three times in working buffer (0.1M Tris, 0.1M potassium chloride) and then incubated with 1 μg/ml of anti-fluorescein-HRP for 30 minutes at room temperature. Following this, the beads were washed three times with working buffer before being resuspended in a final volume of 50 μL of working buffer. The samples were then transferred to a white luminescence 96 well plate and luminescence read in a BMG Fluorostar luminometer following addition of 50 μL Luminol and 50 μL Peroxide (Pierce). Treatments were performed in duplicate, including a heat inactivated control where the terminal transferase was inactivated by heating at 95° C. for 30 minutes.

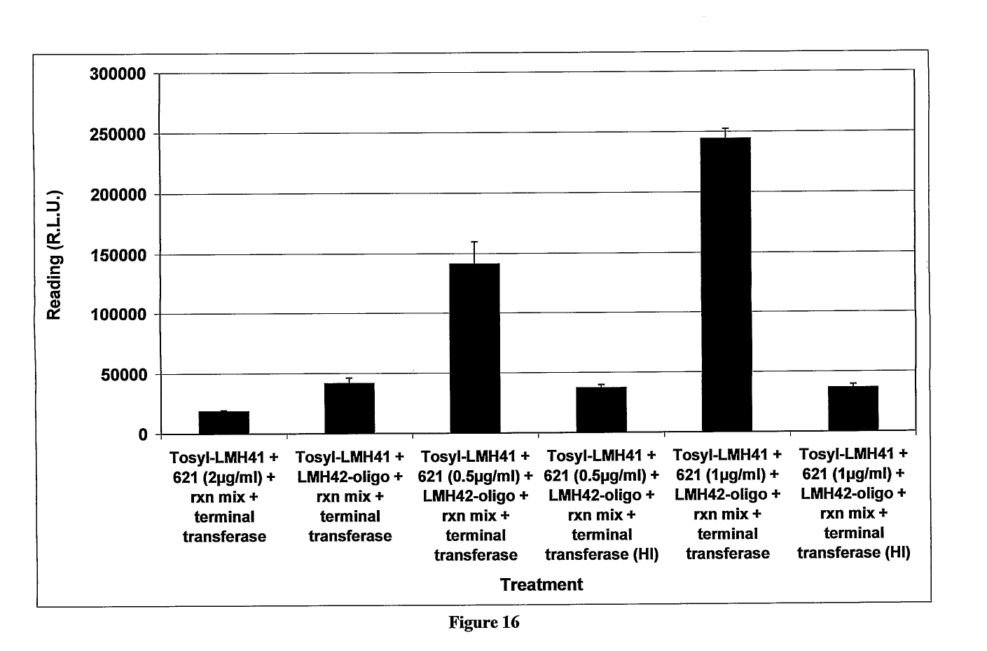

The results show that (FIG. 16) the terminal transferase is able to extend the oligonucleotide substrate and incorporate labelled nucleotides. This extension occurs with the oligonucleotide being complexed to an antibody, which in turn is bound to an antigen which in turn is bound to an antibody conjugated magnetic bead. Thus, in this example the assay is able to detect the target analyte (EGFR).

### Example 10

### Comparison of Telomerase Activity in Crude and Pre-Cleared Extracts

A confluent 10 cm dish containing LAR41 cells was washed twice in situ with 10 ml PBS. Cells were scrapped off the plate in 1 ml PBS and transferred to a 1.5 ml tube. A cell count was performed and the cells centrifuged for 5 minutes at 13,000 r.p.m. The supernatant was removed and RIPA lysis buffer (50 mM Tris, 150 mM NaCl, 1 mM EDTA, 1% Triton X-100, 1% Sodium deoxycholate, 0.1% SDS, protease inhibitor tablet (Roche)) added to the cells (200 μl/1×10⁶ cells). The cells were pipetted up and down a number of times to lyse the cells and then incubated on ice for 30 minutes. Following this, the extracts were centrifuged for at 13,000 r.p.m. for 25 minutes at 4° C. The supernatant was removed to a fresh 1.5 ml tube and analysed for telomerase activity.

Magnetic beads to which the telomerase recognition sequence had been conjugated using amine coupling chemistry (see above) were incubated for 30 minutes at 37° C. in the presence of a reaction buffer (20 mM Tris-HCl, 1.5 mM MgCl₂, 63 mM KCl, 1 mM EGTA, 1 mM EDTA, 150 mM NaCl, 0.005% Tween 20, 12.5 μM biotin-21-dUTP, 18.75 μM dAdG) and 1 μl of an extract derived a bladder cancer cell line (LAR41) that was either crude (no further manipulation of the extract following cell lysis) or pre-cleared by centrifuging the extract at 13,000 r.p.m. (Sorvall Heraeus Fresco Biofuge, rotor 514400) for 25 minutes. The supernatant after centrifuging (pre-cleared extract) was used in side-by-side telomerase extension reactions with the crude extracts.

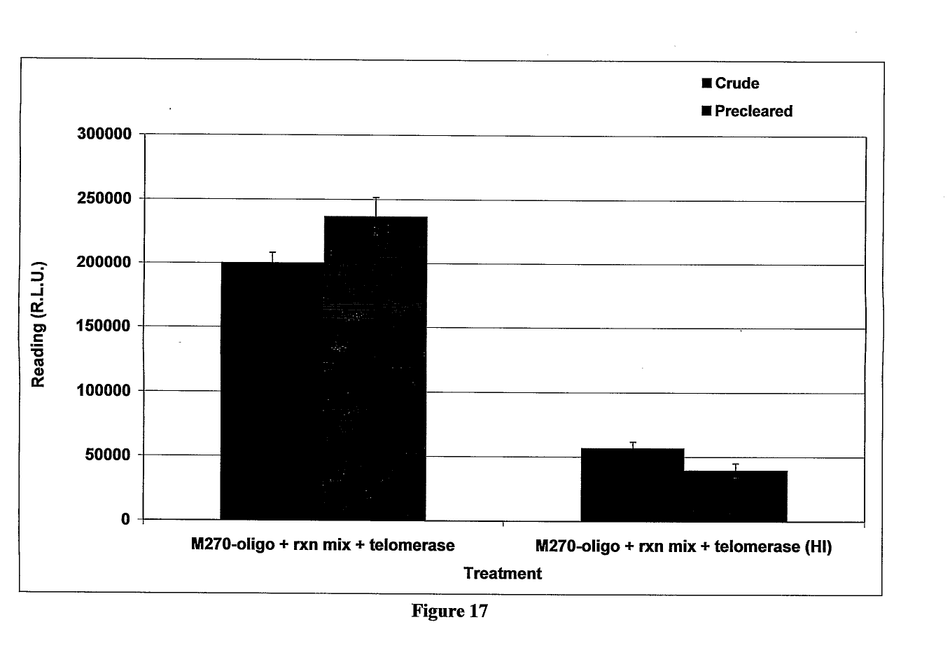

The results are provided in FIG. 17. As controls, heat inactivated (HI) samples are also shown. It is apparent that crude lysed cells which express telomerase can be used as a source of this enzyme for the method of the invention.

### Example 11

### Amplified Protein Luminescence (APL) Assay for the Detection of the CT Antigen NY-ESO-1

Tosyl-activated Dynal Beads are coupled to the anti-NY-ESO-1 mAb E978 (Sugita et al., 2004) using standard amine coupling chemistry (overnight coupling at pH8.3). After coupling the beads are washed well and stored in PBS.

Anti-NY-ESO-1 antibody ES121 (Chitale et al., 2004) is functionalised with the target nucleotide containing the recognition sequence for telomerase (TTTTTTAATCCGTCGAGCAGAGTTAGGGTTAGGGTTAG) using hydrazine chemistry as described previously (Kozlov et al., 2004). The resulting conjugate is purified from residual reactants using size exclusion HPLC on a Superose 12 10/300 column (GE Amersham) using PBS buffer at a flow rate of 1 ml/min and a column temperature of 25° C.

The ability of these two antibodies to bind simultaneously to the NY-ESO-1 has been confirmed using a BIAcore 3000 optical biosensor with the mAb ES978 immobilised on the sensor surface using NHS/EDC chemistry and also by sandwich ELISA.

Active telomerase preparations are generated by lysis of LIM 1215 cells (10⁷ cells per ml) in 0.5% CHAPS, 10 mM Tris-HCl, 1 mM MgCl₂ and 1 mM EGTA (pH7.5) containing 0.5% glycerol and protease inhibitors (MiniProtease, Roche). The activity was confirmed by TRAP assay (Chemicon) and protein levels were determined by BCA analysis Aliquots were stored at −70 C for activation of the telomerase oligonucleotide target in the APL assay.

The APL assay is validated using recombinant NY-ESO-1 (Davis et al, 2004). NY-ESO calibration standards were generated in TC medium by serial dilution.

The mAb E978-Dynal beads (approximately 10⁷ beads) are added to tissue culture medium (up to 1 ml) containing the NY-ESO-1 standards in a 1.5 ml Eppendorf tube. The NY-ESO-1 is selectively recovered onto the coupled Dynal beads by shaking at 25° C. for 1 hour. The beads are then pulled down by a magnetic particle concentrator and washed with PBS. After washing the beads are resuspended in 200 μl of PBS buffer containing the oligonucleotide-functionalised mAb ES121 and a complex formed with the NY-ESO-1, which had been trapped on the Dynal beads, by further shaking at 25° C. for 1 hr. The beads are then pulled down and washed with elongation buffer (20 mM Tris-HCl, 1.5 mM MgCl2, 63 mM KCl, 1 mM EGTA, 0.1 μg/ml containing 0.005% Tween 20 (Xu et al., 2002) before resuspending in elongation buffer containing dATP and dGTP and biotinylated dUTP (200 μl). Telomerase extract from LIM1215 cells (1 μl, see above) is then added and the tube incubated at 32° C. for 30 min with shaking. The beads are then washed 1× elongation buffer followed by a further wash(es) to remove background signals such as by increasing NaCl concentrations, 0.1M NaOH, lowering pH or using low concentrations of detergents, before adding Streptavidin-HRP (1501 μl, 2 μg/ml in PBS) and incubating for 30 min at 32° C. The beads are then washed 5×0.1M Tris-HCl containing 0.1M KCl (pH8.5) before re-suspending in 25 μl of the same buffer for transfer to a 96 well LumiNunc plate.

The LumiNunc plate is transferred to a BMG FluoroStar Luminometer and 50111 of luminol with enhancer and 50 μl H₂O₂ (SuperSignal, Pierce Biotechnology Inc.) added using the automated delivery pumps of the instrument and the luminescence signal recorded.

### Example 12

### Amplified Protein Luminescence (APL) Assay for the Detection of the Anti-CT Antigen Antibodies

Tosyl-activated Dynal Beads were coupled to the recombinant NY-ESO-1 (Davis et al., 2004) using standard amine coupling chemistry (overnight coupling at pH8.3). After coupling the beads were well washed and stored in PBS.

Anti-human anti-IgG goat antibody (Bio-Rad) was functionalised with the target nucleotide sequence for terminal transferase using hydrazine chemistry as described previously (Kozlov et al., 2004). The resulting conjugate was purified from residual reactants using size exclusion HPLC on a Superose 12 10/300 column (GE Amersham) using PBS buffer at a flow rate of 1 ml/min and a column temperature of 25° C.

Urine samples (100 uL) containing potential anti-NY-ESO-1 antibodies were added to white Nunc 96 well plates comprising NY-ESO-1-Dynal beads (approximately 10⁷ beads). The anti-NY-ESO-1 antibodies were selectively recovered onto the Dynal beads by shaking (10,000 rpm) at 25° C. for 1 hour. The beads were then pulled down to the surface of the plate by a magnetic attraction and well washed with PBS. After washing the beads were resuspended in 150 μl of PBS buffer containing the oligonucleotide-functionalised anti-human anti-IgG goat antibody and a complex formed with the anti-NY-ESO-1 antibody, which had been trapped on the Dynal beads, by further shaking (10,000 rpm) at 25° C. for 1 h. The beads are then pulled down to the plate surface and washed with terminal transferase elongation buffer (Genesearch reaction buffer, 2.5 mM CoCl₂) before resuspending in elongation buffer containing dATP and dGTP and biotinylated dUTP (50 μl). Terminal transferase (1 μl, Genesearch, Australia) is then added and the tube incubated at 32° C. for 30 min with shaking (10,000 rpm). The beads are then washed 1× elongation buffer followed by a further wash(es) to remove background signals such as by increasing NaCl concentrations, 0.1M NaOH, lowering pH or using low concentrations of detergents, before adding Streptavidin-HRP (150 μl, 2 μg/ml in PBS) and incubating for 30 min at 32° C. The beads are then washed 5×0.1M Tris-HCl containing 0.1M KCl (pH8.5) before re-suspending in 25 μl of the same buffer for transfer to a 96 well LumiNunc plate.

The LumiNunc plate is transferred to a BMG FluoroStar Luminometer and 50 μl of luminol with enhancer and 50 μl H₂O₂ (SuperSignal, Pierce Biotechnology Inc.) added using the automated delivery pumps of the instrument and the luminescence signal recorded.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Adler et al. (2003) Biochem. Biophys. Res. Commun. 308:240-250.
Ansari et al. (2001) Chem. Biol. 8:583-592.
Arora et al. (2002) J. Am. Chem. Soc. 124:13067-13071.
Bronks et al. (1995) Anal. Chem. 65:2750-2757.
Bryan et al. (1998) Proc Natl Acad Sci USA. 95:8479-8484.
Chitale et al. (2004) Mod Pathol. 23 July [Epub ahead of print].
Chu et al. (1986) Nucl. Acids Res. 14:5591-5603.
Davis et al. (2004) Proc Natl Acad Sci USA. 101: 10697-10702.
Domagala et al. (2000) Growth Factors 18:11-29.
Graham et al. (1977) J. Gen Virol 36: 59-72.
Hendrickson et al. (1995) Nucl. Acids Res. 23:522-529.
Joeger (1995) Clin. Chem. 41:1371-1377.
Jungbluth et al. (2001) Int. J. Cancer: 92:856-860.
Kozlov et al. (2004) Biopolymers 73:621-630.
Kwon et al. (2004) J. Am. Chem. Soc. 126:15940-15941.
Murphy et al. (2005) Prep. Biochem. Biotech. 35:119-134.
Niemeyer et al. (2003) Nucl. Acids Res. 31:e90.
Sano et al. (1992) Science 258:120-122.
Scanlan et al. (2002) Immunol. Rev. 188:22-32.
Schweitzer et al. (2000) Proc Natl Acad Sci USA. 97:10113-10119.
Sperl et al. (1995) J. Immunol. Meth. 186:181-194.
Sugita et al. (2004) Cancer Res. 64:2199-2204.
Whitehead et al. (1985) J Natl Cancer Inst. 74:759-765.
Xu et al. (2002) Clinical Chem. 48:1016-1020.
Zhang et al. (2001) Proc. Natl. Acad. Sci. USA 98:5497-5502.

1. A method of screening for the presence or absence of an analyte in a sample, the method comprising
i) exposing the sample to a first compound that binds the analyte to form an analyte-first compound complex,
ii) exposing the analyte-first compound complex to a second compound which binds the analyte-first compound complex to form an analyte-first compound-second compound complex, wherein the second compound comprises a polynucleotide,
iii) exposing the analyte-first compound-second compound complex to a polymerase under conditions which allow either a) the polymerase to extend the polynucleotide, and/or b) the polymerase to synthesize a complementary strand of the polynucleotide, and
iv) detecting the product of parts a) or b) of step iii),
wherein part b) of step iii) does not comprise using the complementary strand as a template for further polynucleotide synthesis.

2. A method of screening for the presence or absence of an analyte in a sample, the method comprising
i) exposing the sample to a second compound that binds the analyte to form an analyte-second compound complex, wherein the second compound comprises a polynucleotide,
ii) exposing the analyte-second compound complex to a first compound which binds the analyte-second compound complex to form an analyte-first compound-second compound complex,
iii) exposing the analyte-first compound-second compound complex to a polymerase under conditions which allow either a) the polymerase to extend the polynucleotide, and/or b) the polymerase to synthesize a complementary strand of the polynucleotide, and
iv) detecting the product of parts a) or b) of step iii),
wherein part b) of step iii) does not comprise using the complementary strand as a template for further polynucleotide synthesis.

3. The method of claim 1, wherein the polymerase extends a single stranded polynucleotide or a single stranded overhang of a partially double stranded polynucleotide.

4. The method of claim 3, wherein the polymerase is selected from the group consisting of: poly(A)polymerase, telomerase and terminal transferase.

5-6. (canceled)

7. The method of claim 1, wherein the polymerase is a DNA polymerase selected from the group consisting of: Taq polymerase, bacteriophage T4 polymerase, bacteriophage T7 polymerase, and E. coli DNA polymerase I Klenow fragment.

8. The method according to claim 1, wherein the first compound and/or second compound is attached to a solid support.

9. (canceled)

10. The method according to claim 1, wherein step iii) is performed in the presence of at least one detectably labelled nucleotide.

11. (canceled)

12. The method according to claim 1, wherein step iii) is performed in the presence of at least one hapten conjugated nucleotide, wherein the hapten is capable of binding a ligand.

13-32. (canceled)

33. The method according to claim 1, wherein the second compound is an antibody-polynucleotide conjugate.

34. The method according to claim 1, wherein the second compound binds the analyte.

35. The method according to claim 1, wherein the analyte is a marker of a disease state.

36. (canceled)

37. The method according to claim 1, wherein the analyte is a protein or peptide.

38. The method of claim 1, wherein the method comprises
i) exposing the sample to a first compound that binds the analyte to form an analyte-first compound complex, wherein the first compound is attached to a solid support,
ii) exposing the analyte-first compound complex to a second compound which binds the analyte to form an analyte-first compound-second compound complex, wherein the second compound comprises a polynucleotide which can be extended by a telomerase,
iii) exposing the analyte-first compound-second compound complexes to a telomerase in the presence of at least one hapten conjugated nucleotide under conditions which allow the telomerase to extend the polynucleotide,
iv) exposing the products of telomerase activity to a enzyme labelled ligand under conditions which allow the hapten to bind to the ligand,
v) incubating the polynucleotide-hapten-ligand-enzyme complex in the presence of a substrate of the enzyme, and
vi) detecting a detectable signal produced by the activity of the enzyme on the substrate.

39. The method of claim 1, wherein the method comprises
i) exposing, the sample to a first compound that binds the analyte to form an analyte-first compound complex, wherein the first compound is attached to a solid support,
ii) exposing the analyte-first compound complex to a second compound which binds the analyte to form an analyte-first compound-second compound complex, wherein the second compound comprises a polynucleotide which can be extended by a terminal transferase,
iii) exposing the analyte-first compound-second compound complexes to a terminal transferase in the presence of at least one hapten conjugated nucleotide under conditions which allow the terminal transferase to extend the polynucleotide,
iv) exposing the products of terminal transferase activity to a enzyme labelled ligand under conditions which allow the hapten to bind to the ligand,
v) incubating the polynucleotide-hapten-ligand-enzyme complex in the presence of a substrate of the enzyme, and
vi) detecting a detectable signal produced by the activity of the enzyme on the substrate.

40. A method of screening for the presence or absence of an analyte in a sample, the method comprising
i) exposing the sample to a first compound that binds the analyte to form an analyte-first compound complex,
ii) exposing the analyte-first compound complex to a second compound which binds the analyte-first compound complex to form an analyte-first compound-second compound complex,
iii) exposing the analyte-first compound-second compound complex to a third compound which binds the second compound, wherein the third compound comprises a polynucleotide,
iv) exposing the analyte-first compound-second compound-third compound complex to a polymerase under conditions which allow either a) the polymerase to extend the polynucleotide, and/or b) the polymerase to synthesize a complementary strand of the polynucleotide, and
v) detecting the product of parts a) or b) of step iv), wherein part b) of step iv) does not comprise using the complementary strand as a template for further polynucleotide synthesis, and wherein the first and/or second compound is bound to a solid support.

41. A protein-DNA conjugate, wherein the DNA comprises a sequence which can bind, and be extended by, a telomerase, and wherein the protein is covalently attached to the DNA.

42. (canceled)

43. A kit comprising the protein-DNA conjugate of claim 41.

44. A kit comprising a polymerase, and at least two compounds which bind the same analyte, wherein one of said compounds comprises a polynucleotide, wherein the polymerase extends a single stranded polynucleotide or a single stranded overhang of a partially double stranded polynucleotide.

45. (canceled)

46. The kit of claim 44, wherein the polymerase is telomerase or terminal transferase.

47. (canceled)
